(19) United States
(12) Patent Application Publication
(10) Pub. No.: US 2026/0112500 A1
(43) Pub. Date: Apr. 23, 2026

(54) METHOD AND SYSTEM FOR PREDICTING FELINE HYPERTROPHIC CARDIOMYOPATHY DISEASE RISK

(71) Applicant: IDEXX Laboratories, Inc., Westbrook, ME (US)

(72) Inventor: Mary Alexis SEGUIN, North Freedom, WI (US)

(21) Appl. No.: 19/358,903

(22) Filed: Oct. 15, 2025

Related U.S. Application Data
(60) Provisional application No. 63/708,390, filed on Oct. 17, 2024.

(51) Int. Cl. G16H 50/30; G06N 20/20; G16H 10/20; G16H 20/00; G16H 50/20; G16H 50/70

(52) U.S. Cl. CPC G16H 50/30; G06N 20/20; G16H 10/20; G16H 20/00; G16H 50/20; G16H 50/70

(57) ABSTRACT

Systems and methods for assessing the risk of feline hypertrophic cardiomyopathy are described. An ensemble of diagnostic models is trained on a first set of medical training data. A knowledge based diagnostic model is trained on a second set of medical training data. When new patient data is received, the ensemble of diagnostic models is used to determine whether a risk for feline hypertrophic cardiomyopathy is indicated for the new patient data. If the ensemble of diagnostic models indicates a risk for feline hypertrophic cardiomyopathy for the new patient data, the risk of feline hypertrophic cardiomyopathy is assessed using the knowledge based diagnostic model. The knowledge based diagnostic model interacts with a user interface is used to acquire additional information from a clinician. The user interface provides an assessment of the risk of feline hypertrophic cardiomyopathy for the new patient data, and guidance for further testing and treatment.

### CROSS REFERENCE TO RELATED APPLICATIONS

This application claims priority to U.S. Provisional Application No. 63/708,390, filed Oct. 17, 2024, the contents of which are incorporated herein, in their entirety, by reference.

### TECHNICAL FIELD

The present disclosure generally relates to diagnostic models for human and veterinary applications, and more specifically relates to machine learning models for analyzing and predicting the risk of hypertrophic cardiomyopathy in feline patients.

### BACKGROUND

Feline hypertrophic cardiomyopathy (HCM) is a common heart disease in cats who have severe thickening (hypertrophy) in the walls of the left ventricle. In primary HCM, hypertrophy of the left ventricle is related to a genetic mutation that causes the sarcomere to be more responsive to calcium than normal, resulting in hypercontractility of the myocardium. However, the mechanism of how this dysfunction causes hypertrophy of the left ventricle is unclear. This thickening of the left ventricle wall impairs the normal function of the left heart during diastole. Cats with HCM typically appear clinically healthy with no obvious symptoms. Cats with more advanced HCM may present with clinical symptoms of congestive heart failure or thromboembolic disease.

In most cases with primary HCM, the left atrium is also enlarged due to the decreased volume of the left ventricle, resulting in a diminished ability of the heart to supply blood and oxygen to the body. This decreased efficiency of the heart contributes to the formation of blood clots in the heart, cardiac arrythmias, and heart failure. These markers are generally the first clinical signs that are observed in feline patients with HCM. In less advanced cases of feline HCM, clinical signs may be subtle or not present at all. Some cats with HCM may have a heart murmur or abnormality that prompt a cardiac evaluation, but some cats with heart disease have normal-sounding hearts. Additionally, healthy cats have also been found to have physiologic heart murmurs that are not indicative of an underlying heart disease, making it an unreliable method of screening for potential HCM in cats.

The lack of clinical signs and the absence of adequate screening for the disease make it very difficult to diagnose feline HCM at an early stage, where interventions and therapies are most effective. Often, feline HCM is determined via echocardiography showing left ventricular walls thicker than 6 mm. However, this sign alone does not indicate primary HCM as it requires a diagnosis of exclusion. In order to definitively diagnose HCM, blood tests and blood pressure assessments should be performed to eliminate the possibility of hyperthyroidism and hypertension, the two most common causes of left ventricular wall hypertrophy. In general practice, diagnosing HCM requires a lot of time and resources and will typically only occur if there are strong clinical indicators that HCM may be present. If a patient is consistently exhibiting clinically healthy signs, there is no reason for a veterinarian to suspect HCM and spend considerable resources testing for it.

Veterinarians generally rely on abnormalities in a physical exam to recognize the possibility of feline HCM. Additional diagnostic/laboratory testing is required to definitively diagnose HCM. Veterinarians are likely to rely on abnormal heart rhythms/sounds or advanced complications (e.g., congestive heart failure) as a primary trigger to consider HCM as a differential diagnosis. However, cardiac arrythmias and heart murmurs are inconsistently seen in cats with HCM, and advanced complications may only be present when the patient is in crisis. Veterinarians are unlikely to order an echocardiogram for HCM if there are no cardiac abnormalities present, and HCM may go unrecognized until it results in an acute crisis. These crises can be severe, requiring hospitalization, and can result in sudden death. The ability to detect feline HCM prior to the development of an acute crisis is essential to instituting lifesaving treatment before the patient has a critical complication.

Since there are very few initial symptoms of HCM and they are not always present, cats with HCM may appear healthy and remain undiagnosed. As it is easy for a veterinarian to miss the signs of HCM, especially in the early stages, there exists a need for a new approach that utilizes machine learning to assess the risk for HCM at an early stage of the disease, using basic laboratory tests and clinical observations.

### SUMMARY

At least the above-discussed needs are addressed, and technical solutions are achieved in the art, by various embodiments disclosed herein.

Some embodiments pertain to a method executed by a programmed data processing device system comprising receiving first medical training data; training a plurality of first machine learning models on the received first medical training data to generate an ensemble of diagnostic models; receiving second medical training data; training a second machine learning model on the received second medical training data to generate a knowledge based diagnostic model; receiving new patient data; determining whether the ensemble of diagnostic models indicates a risk for feline hypertrophic cardiomyopathy for the new patient data; and, in a case where the ensemble of diagnostic models indicates a risk for feline hypertrophic cardiomyopathy for the new patient data, assessing the risk of feline hypertrophic cardiomyopathy using the knowledge based diagnostic model.

In some embodiments, the ensemble of diagnostic models includes one or more of a decision tree model, a random forest model, a neural network, a support vector machine, or a nearest neighbor classifier.

In some embodiments, training the plurality of first machine learning models includes selecting at least a first machine learning model and a second machine learning model for the ensemble of diagnostic models, wherein the second machine learning model is different from the first machine learning model; training the first machine learning model using a first subset of the received first medical training data to generate a first model of the ensemble of diagnostic models; and training the second machine learning model using a second subset of the received first medical training data to generate a second model of the ensemble of diagnostic models, wherein the second subset of the received first medical training data is different from the first subset of the received first medical training data. The risk for feline hypertrophic cardiomyopathy for the new patient data is determined based on outputs of the first machine learning model and the second machine learning model. In some embodiments, it is determined that the ensemble of diagnostic models indicates a risk for feline hypertrophic cardiomyopathy for the new patient data in a case where the outputs of either the first machine learning model or the second machine learning indicate the risk for feline hypertrophic cardiomyopathy for the new patient data.

In some embodiments, training the second machine learning model includes generating an expert model including a plurality of rules based on domain knowledge included in the received second medical training data; refining the plurality of rules in the expert model based on patient data included in the received second medical training data; and generating the knowledge based diagnostic model based on the refined plurality of rules.

In some embodiments, the knowledge based diagnostic model includes one or more of a rules based model and a data driven machine learning model.

In some embodiments, assessing the risk of feline hypertrophic cardiomyopathy using the knowledge based diagnostic model further includes displaying, on a user interface, a series of prompts to receive further patient medical data; and assessing the risk of feline hypertrophic cardiomyopathy based the received further patient medical data in response to the series of prompts. In some embodiments, each subsequent prompt of the series of prompts is dynamically selected using the knowledge based diagnostic model based on a response to a preceding prompt of the series of prompts.

In some embodiments, the knowledge based diagnostic model is an interactive model, and assessing the risk of feline hypertrophic cardiomyopathy includes interacting with a user using the knowledge based diagnostic model to provide guidance on diagnosis and treatment of feline hypertrophic cardiomyopathy.

In some embodiments, a diagnostic system for assessing the risk of feline hypertrophic cardiomyopathy comprises a memory configured to store instructions, and a processor communicatively connected to the memory and configured to execute the stored instructions.

The processor executes the stored instructions to receive first medical training data; train a plurality of first machine learning models on the received first medical training data to generate an ensemble of diagnostic models; receive second medical training data; train a second machine learning model on the received second medical training data to generate a knowledge based diagnostic model; receive new patient data; determine whether the ensemble of diagnostic models indicates a risk for feline hypertrophic cardiomyopathy for the new patient data; and, in a case where the ensemble of diagnostic models indicates a risk for feline hypertrophic cardiomyopathy for the new patient data, assess the risk of feline hypertrophic cardiomyopathy using the knowledge based diagnostic model.

In some embodiments, a non-transitory computer readable storage medium is configured to store a program that executes the diagnostic methods discussed above.

In some embodiments, a method, executed by a programmed data processing device system, of assessing the risk of feline hypertrophic cardiomyopathy comprises receiving new patient data; receiving a machine-learning based diagnostic model and a knowledge based diagnostic model; determining whether the machine-learning based diagnostic model indicates a risk for feline hypertrophic cardiomyopathy for the new patient data; and, in a case where the machine-learning based diagnostic model indicates a risk for feline hypertrophic cardiomyopathy for the new patient data, assessing the risk of feline hypertrophic cardiomyopathy using the knowledge based diagnostic model.

In some embodiments, the machine-learning based diagnostic model includes one or more of a decision tree model, a random forest model, a neural network, a support vector machine, or a nearest neighbor classifier.

In some embodiments, the method further comprises receiving first training data; and training the machine-learning based diagnostic model by selecting at least a first machine learning model and a second machine learning model, wherein the second machine learning model is different from the first machine learning model; training the first machine learning model using a first subset of the received first training data to generate a first diagnostic model; training the second machine learning model using a second subset of the received first training data to generate a second diagnostic model; and combining the first diagnostic model and the second diagnostic model into an ensemble diagnostic model to be used as the machine-learning based diagnostic model, wherein the risk for feline hypertrophic cardiomyopathy for the new patient data is determined based on an output of the ensemble diagnostic model.

In some embodiments, it is determined that the ensemble diagnostic model indicates a risk for feline hypertrophic cardiomyopathy for the new patient data in a case where the outputs of either the first diagnostic model or the second diagnostic model indicate the risk for feline hypertrophic cardiomyopathy for the new patient data.

In some embodiments, the method further comprises receiving second training data; and training the knowledge based diagnostic model by generating an expert model including a plurality of rules based on domain knowledge included in the received second medical training data; and refining the plurality of rules in the expert model based on patient data included in the received second medical training data.

In some embodiments, the knowledge based diagnostic model includes one or more of a rule based model or a data driven machine learning model.

In some embodiments, assessing the risk of feline hypertrophic cardiomyopathy using the knowledge based diagnostic model further includes displaying, on a user interface, a series of prompts to receive further patient medical data; and assessing the risk of feline hypertrophic cardiomyopathy based the received further patient medical data in response to the series of prompts.

In some embodiments, each subsequent prompt of the series of prompts is dynamically selected using the knowledge based diagnostic model based on a response to a preceding prompt of the series of prompts.

In some embodiments, the knowledge based diagnostic model is an interactive model, and assessing the risk of feline hypertrophic cardiomyopathy includes interacting with a user using the knowledge based diagnostic model to provide guidance on diagnosis and treatment of feline hypertrophic cardiomyopathy.

In some embodiments, a diagnostic system for assessing the risk of feline hypertrophic cardiomyopathy comprises a memory configured to store instructions; and a processor communicatively connected to the memory and configured to execute the stored instructions to receive new patient data; receive a machine-learning based diagnostic model and a knowledge based diagnostic model; determine whether the machine-learning based diagnostic model indicates a risk for feline hypertrophic cardiomyopathy for the new patient data; and, in a case where the machine-learning based diagnostic model indicates a risk for feline hypertrophic cardiomyopathy for the new patient data, assess the risk of feline hypertrophic cardiomyopathy using the knowledge based diagnostic model.

In some embodiments, the system further executes the other aspects of the methods described above.

In some embodiments, a non-transitory computer readable storage medium is configured to store a program that executes the various aspects of the diagnostic methods described above. Subsets or combinations of various embodiments described above provide further embodiments.

### BRIEF DESCRIPTION OF THE DRAWINGS

It is to be understood that the attached drawings are for purposes of illustrating aspects of various embodiments and may include elements that are not to scale. It is noted that like reference characters in different figures refer to the same objects.

FIG. 1 shows a computing device system, according to some embodiments.

FIG. 2 shows another computing device system, according to some embodiments.

FIG. 3 shows a block diagram of a diagnostic system for assessing the risk of feline hypertrophic cardiomyopathy, according to some embodiments.

FIG. 4 shows a flowchart of a machine learning based feline hypertrophic cardiomyopathy risk assessment method, according to some embodiments.

FIG. 5 shows a flowchart of a method of generating machine learning models for assessing the risk of feline hypertrophic cardiomyopathy, according to some embodiments.

FIG. 6 shows a flowchart of an interactive machine learning based feline hypertrophic cardiomyopathy risk assessment method, according to some embodiments.

FIG. 7A shows an analyzer, according to some embodiments.

FIG. 7B shows a slide processing assembly for an analyzer, according to some embodiments.

FIG. 7C shows a slide for an analyzer, according to some embodiments.

FIG. 8 shows a schematic for a control system for an analyzer, according to some embodiments.

### DETAILED DESCRIPTION

In some embodiments, the computer systems described herein execute methods for implementing machine learning models that predict the risk of feline hypertrophic cardiomyopathy. It should be noted that the aspects or embodiments of the present disclosure are not limited to these or any other examples provided herein, which are referred to for purposes of illustration only.

In this regard, in the descriptions herein, certain specific details are set forth in order to provide a thorough understanding of various embodiments. However, one skilled in the art will understand that the invention may be practiced at a more general level without one or more of these details. In other instances, well-known structures have not been shown or described in detail to avoid unnecessarily obscuring descriptions of various embodiments.

Any reference throughout this specification to one “aspect” or “embodiment,” an “aspect” or “embodiment,” an example “aspect” or “embodiment,”, an illustrated “aspect” or “embodiment,” a particular “aspect” or “embodiment,” or the like means that a particular feature, structure, or characteristic described in connection with the aspect or embodiment is included in at least one or more aspects or embodiments. Thus, any appearance of the phrase in one “aspect” or “embodiment,” in an “aspect” or “embodiment,” in an example “aspect” or “embodiment,” in this illustrated “aspect” or “embodiment,” in this particular “aspect” or “embodiment,” or the like in this specification is not necessarily all referring to one aspect or embodiment or a same aspect or embodiment. Furthermore, the particular features, structures or characteristics of different aspects or embodiments of the disclosure may be combined in any suitable manner to form one or more other aspects or embodiments of the disclosure. Further, the term aspect or embodiment may be used interchangeably.

Unless otherwise explicitly noted or required by context, the word “or” is used in this disclosure in a non-exclusive sense. In addition, unless otherwise explicitly noted or required by context, the word “set” is intended to mean one or more. For example, the phrase, “a set of objects”means one or more of the objects.

In the following description, some embodiments may be implemented at least in part by a data processing device system configured by a software program. Such a program may equivalently be implemented as multiple programs, and some or all of such software program(s) may be equivalently constructed in hardware.

Further, the phrase “at least” is or may be used herein at times merely to emphasize the possibility that other elements may exist beside those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase, “based at least on A” includes A as well as the possibility of one or more other additional elements besides A. In the same manner, the phrase, “based on A” includes A, as well as the possibility of one or more other additional elements besides A. However, the phrase, “based only on A” includes only A. Similarly, the phrase “configured at least to A” includes a configuration to perform A, as well as the possibility of one or more other additional actions besides A. In the same manner, the phrase “configured to A” includes a configuration to perform A, as well as the possibility of one or more other additional actions besides A. However, the phrase, “configured only to A” means a configuration to perform only A.

The word “device,” the word “machine,” the word “system,” and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified according to various embodiments that a device or machine or device system resides entirely within a same housing to exclude embodiments where the respective device, machine, system, or device system resides across different housings. The word “device” may equivalently be referred to as a “device system”in some embodiments.

The phrase “derivative thereof” and the like is or may be used herein at times in the context of a derivative of data or information merely to emphasize the possibility that such data or information may be modified or subject to one or more operations. For example, if a device generates first data for display, the process of converting the generated first data into a format capable of being displayed may alter the first data. This altered form of the first data may be considered a derivative of the first data. For instance, the first data may be a one-dimensional array of numbers, but the display of the first data may be a color-coded bar chart representing the numbers in the array. For another example, if the above-mentioned first data is transmitted over a network, the process of converting the first data into a format acceptable for network transmission or understanding by a receiving device may alter the first data. As before, this altered form of the first data may be considered a derivative of the first data. For yet another example, generated first data may undergo a mathematical operation, a scaling, or a combining with other data to generate other data that may be considered derived from the first data. In this regard, it can be seen that data is commonly changing in form or being combined with other data throughout its movement through one or more data processing device systems, and any reference to information or data herein is intended to include these and like changes, regardless of whether or not the phrase “derivative thereof” or the like is used in reference to the information or data, unless otherwise required by context. As indicated above, usage of the phrase “or a derivative thereof” or the like merely emphasizes the possibility of such changes. Accordingly, the addition of or deletion of the phrase “or a derivative thereof” or the like should have no impact on the interpretation of the respective data or information. For example, the above-discussed color-coded bar chart may be considered a derivative of the respective first data or may be considered the respective first data itself.

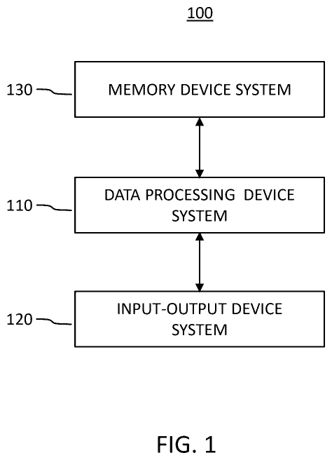

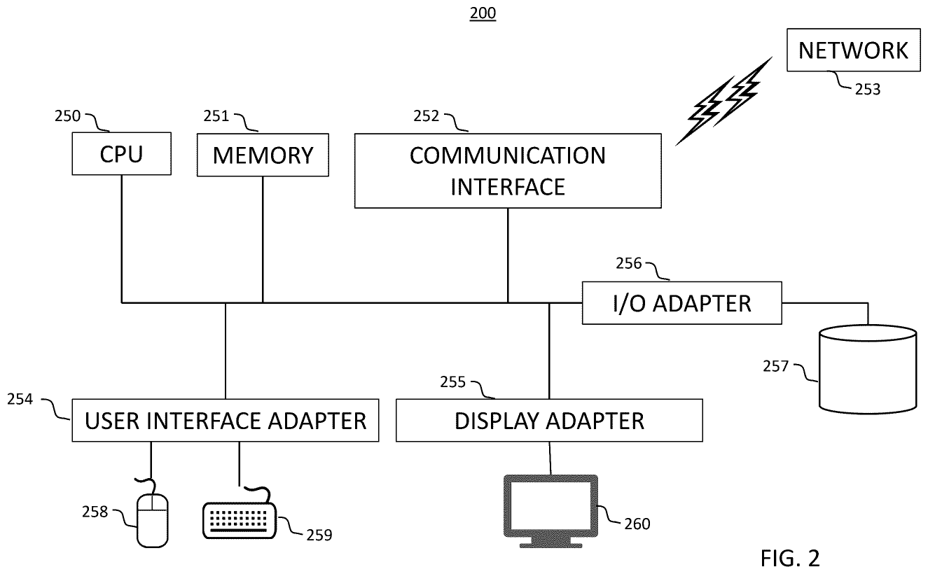

The term “program” in this disclosure should be interpreted to include one or more programs including a set of instructions or modules that may be executed by one or more components in a system, such as a controller system or data processing device system, to cause the system to perform one or more operations. The set of instructions or modules may be stored by any kind of memory device, such as those described subsequently with respect to the memory device system 130, 251, or both, shown in FIGS. 1 and 2, respectively. In addition, this disclosure may describe or similarly describe that the instructions or modules of a program are configured to cause the performance of an action. The phrase “configured to” in this context is intended to include at least (a) instructions or modules that are presently in a form executable by one or more data processing devices to cause performance of the action (e.g., in the case where the instructions or modules are in a compiled and unencrypted form ready for execution), and (b) instructions or modules that are presently in a form not executable by the one or more data processing devices, but could be translated into the form executable by the one or more data processing devices to cause performance of the action (e.g., in the case where the instructions or modules are encrypted in a non-executable manner, but through performance of a decryption process, would be translated into a form ready for execution). Such descriptions should be deemed to be equivalent to describing that the instructions or modules are configured to cause the performance of the action. The word “module” may be defined as a set of instructions. The word “program” and the word “module” may each be interpreted to include multiple sub-programs or multiple sub-modules, respectively. In this regard, reference to a program or a module may be considered to refer to multiple programs or multiple modules.

Further, it is understood that information or data may be operated upon, manipulated, or converted into different forms as it moves through various devices or workflows. In this regard, unless otherwise explicitly noted or required by context, it is intended that any reference herein to information or data includes modifications to that information or data. For example, “data X” may be encrypted for transmission, and a reference to “data X” is intended to include both its encrypted and unencrypted forms, unless otherwise required or indicated by context. However, non-usage of the phrase “or a derivative thereof” or the like nonetheless includes derivatives or modifications of information or data just as usage of such a phrase does, as such a phrase, when used, is merely used for emphasis.

Further, the phrase “graphical representation” used herein is intended to include a visual representation presented via a display device system and may include computer-generated text, graphics, animations, or one or more combinations thereof, which may include one or more visual representations originally generated, at least in part, by an image-capture device.

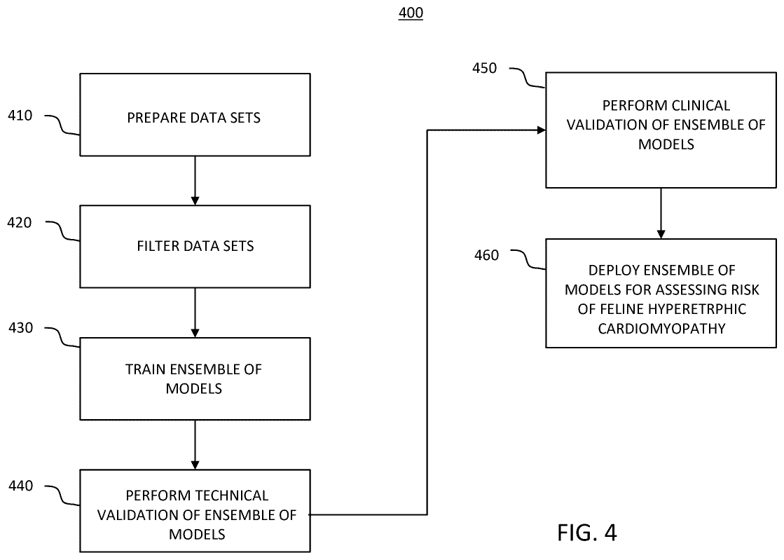

Further still, example methods are described herein with respect to FIGS. 4-6. Such figures are described to include blocks associated with computer-executable instructions. It should be noted that the respective instructions associated with any such blocks herein need not be separate instructions and may be combined with other instructions to form a combined instruction set. The same set of instructions may be associated with more than one block. In this regard, the block arrangements shown in method FIGS. 4-6 herein are not limited to an actual structure of any program or set of instructions or required ordering of method tasks, and such method FIGS. 4-6, according to some embodiments, merely illustrate the tasks that instructions are configured to perform, for example upon execution by a data processing device system in conjunction with interactions with one or more other devices or device systems.

FIG. 1 schematically illustrates a system 100 according to some embodiments. In some embodiments, the system 100 may be a computing device 200 (as shown in FIG. 2). In some embodiments, the system 100 includes a data processing device system 110, an input-output device system 120, and a processor-accessible memory device system 130. The processor-accessible memory device system 130 and the input-output device system 120 are communicatively connected to the data processing device system 110.

The data processing device system 110 includes one or more data processing devices that implement or execute, in conjunction with other devices, such as one or more of those in the system 100, control programs associated with some of the various embodiments. Each of the phrases “data processing device,” “data processor,” “processor,” and “computer” is intended to include any data processing device, such as a central processing unit (“CPU”), a circuit, a field programmable gate array (FPGA), a desktop computer, a laptop computer, a mainframe computer, a tablet computer, a personal digital assistant, a cellular phone, and any other device configured to process data, manage data, or handle data, whether implemented with electrical, magnetic, optical, biological components, or the like.

The memory device system 130 includes one or more processor-accessible memory devices configured to store information, including the information needed to execute the control programs associated with some of the various embodiments. The memory device system 130 may be a distributed processor-accessible memory device system including multiple processor-accessible memory devices communicatively connected to the data processing device system 110 via a plurality of computers and/or devices. On the other hand, the memory device system 130 need not be a distributed processor-accessible memory system and, consequently, may include one or more processor-accessible memory devices located within a single data processing device.

Each of the phrases “processor-accessible memory” and “processor-accessible memory device” is intended to include any processor-accessible data storage device, whether volatile or nonvolatile, electronic, magnetic, optical, or otherwise, including but not limited to, registers, floppy disks, hard disks, Compact Discs, DVDs, flash memories, ROMs (Read-Only Memory), and RAMs (Random Access Memory). In some embodiments, each of the phrases “processor-accessible memory” and “processor-accessible memory device” is intended to include a non-transitory computer-readable storage medium. In some embodiments, the memory device system 130 can be considered a non-transitory computer-readable storage medium system.

The phrase “communicatively connected” is intended to include any type of connection, whether wired or wireless, between devices, data processors, or programs in which data may be communicated. Further, the phrase “communicatively connected” is intended to include a connection between devices or programs within a single data processor, a connection between devices or programs located in different data processors, and a connection between devices not located in data processors at all. In this regard, although the memory device system 130 is shown separately from the data processing device system 110 and the input-output device system 120, one skilled in the art will appreciate that the memory device system 130 may be located completely or partially within the data processing device system 110 or the input-output device system 120. Further in this regard, although the input-output device system 120 is shown separately from the data processing device system 110 and the memory device system 130, one skilled in the art will appreciate that such system may be located completely or partially within the data processing system 110 or the memory device system 130, depending upon the contents of the input-output device system 120. Further still, the data processing device system 110, the input-output device system 120, and the memory device system 130 may be located entirely within the same device or housing or may be separately located, but communicatively connected, among different devices or housings. In the case where the data processing device system 110, the input-output device system 120, and the memory device system 130 are located within the same device, the system 100 of FIG. 1 can be implemented by a single application-specific integrated circuit (ASIC) in some embodiments.

The input-output device system 120 may include a mouse, a keyboard, a touch screen, another computer, or any device or combination of devices from which a desired selection, desired information, instructions, or any other data is input to the data processing device system 110. The input-output device system 120 may include any suitable interface for receiving information, instructions or any data from other devices and systems described in various ones of the embodiments.

The input-output device system 120 also may include an image generating device system, a display device system, a speaker device system, a processor-accessible memory device system, or any device or combination of devices to which information, instructions, or any other data is output from the data processing device system 110. In this regard, if the input-output device system 120 includes a processor-accessible memory device, such memory device may or may not form part or all of the memory device system 130. The input-output device system 120 may include any suitable interface for outputting information, instructions or data to other devices and systems described in various ones of the embodiments. In this regard, the input-output device system may include various other devices or systems described in various embodiments.

FIG. 2 shows an example of a computing device system 200, according to some embodiments. The computing device system 200 may include a processor 250, corresponding to the data processing device system 110 of FIG. 1, in some embodiments. The memory 251, input/output (I/O) adapter 256, and non-transitory storage medium 257 may correspond to the memory device system 130 of FIG. 1, according to some embodiments. The user interface adapter 254, mouse 258, keyboard 259, display adapter 255, and display 260 may correspond to the input-output device system 120 of FIG. 1, according to some embodiments. The computing device 200 may also include a communication interface 252 that connects to a network 253 for communicating with other computing devices.

Referring to FIGS. 1, 2, and 4-6 various methods 400, 500, and 600 may be performed by way of associated computer-executable instructions according to some example embodiments. In various example embodiments, a memory device system (e.g., memory device system 130/memory 251) is communicatively connected to a data processing device system (e.g., data processing device systems 110/processor 250) and stores a program executable by the data processing device system to cause the data processing device system to execute various aspects of methods 400, 500, and 600 via interaction with at least, for example, various databases. In these various embodiments, the program may include instructions configured to perform, or cause to be performed, various ones of the instructions associated with execution of various aspects of methods 400, 500, and 600. In some embodiments, methods 400, 500, and 600 may include a subset of the associated blocks or additional blocks than those shown in FIGS. 4-6. In some embodiments, methods 400-600 may include a different sequence indicated between various ones of the associated blocks shown in FIGS. 4-6.

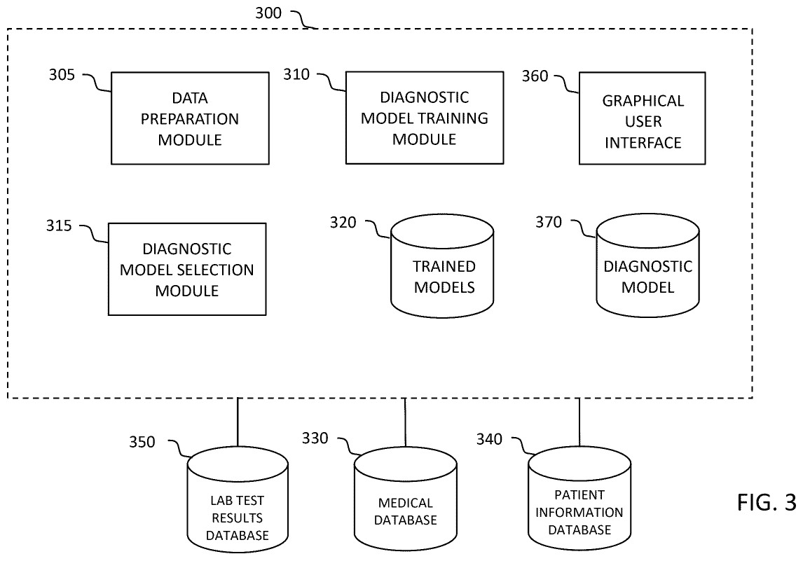

FIG. 3 shows an example of a diagnostic system 300 for assessing the risk of feline hypertrophic cardiomyopathy, according to some embodiments. The system 300 may be a particular implementation of the systems 100, 200 according to some aspects. In some embodiments, the diagnostic system 300 is implemented by programmed instructions stored in one or more memories and executed by one or more processors of the systems 100, 200.

In some embodiments, the diagnostic system 300 includes a data preparation module 305, a diagnostic model training module 310, a diagnostic model validation/selection module 315, and one or more diagnostic models 320. In some embodiments, the diagnostic system 300 may be communicatively connected to one or more databases 330, 340, and 350. In some embodiments, the diagnostic system 300 includes a graphical user interface 360 to permit a user to interact with the system 300. In some embodiments, the diagnostic system 300 includes a knowledge based third diagnostic model 370 which interacts with the user via the graphical user interface 360 to provide advanced guidance and diagnosis information. In some embodiments, the one or more diagnostic models 320 may be stored in a database.

In some embodiments, a medical database 330 stores reference medical data such as ranges of normal, low, and high test results for various diagnostic tests performed in the veterinary clinic or at a veterinary reference laboratory using various diagnostic testing instruments. In some embodiments, the diagnostic tests may be performed by mobile laboratories, using home testing kits, etc. In some embodiments, the diagnostic system 300 may access the medical database 330 to compare actual patient test results, stored in a laboratory test results database 350, with the typical ranges stored in the medical database 330 to interpret the test results performed at the veterinary clinic, the veterinary reference laboratories or using other means. In some embodiments, the diagnostic tests include complete blood count (CBC), blood chemistry, PCR assays etc.

In some embodiments, a patient information database 340 stores a patient's medical record, including patient demographic information, vital signs at each clinical visit, diagnoses, medications, treatment plans, progress notes, patient problems, vaccine history, test results, and imaging data such as radiographs. The demographic data may include species, breed, weight, age, gender, and geographic location, for example. In some embodiments, the medical record may also include information on test results (for example, CBC, blood chemistry, pathology, urinalysis, serology, and PCR (polymerase chain reaction) panels/assays), vector of exposure, and diagnoses, obtained from the laboratory test results database 350.

In some embodiments, the diagnostic system 300 is deployed on a point of care (POC) terminal located at a veterinarian's office or a clinic. The POC terminal may be connected to the veterinary reference laboratories or the diagnostic testing instruments at the veterinary clinic to receive test results for a patient. In some embodiments, the POC terminal may be connected to one or more software servers. In some embodiments, the software servers provide centralized software development resources and support for generating machine learning models (diagnostic models 320 and 370) for assessing the risk of feline hypertrophic cardiomyopathy. In some embodiments, the diagnostic models 320 and 370 may be deployed and executed locally on the POC terminal. In other embodiments, the diagnostic system 300 may be deployed on the server, with the POC terminal acting as a “client” that connects to the server, which executes the diagnostic models 320 and 370 based on patient information transmitted to the server from the POC terminal.

In some embodiments, the diagnostic models 320 are used to provide alerts to clinicians when a patient has an increased likelihood of feline hypertrophic cardiomyopathy. In some embodiments, further targeted screening may be performed using diagnostic model 370, in response to the alert, to validate the presence/absence of feline hypertrophic cardiomyopathy. This approach provides significant advantages for recognizing and treating feline hypertrophic cardiomyopathy by reducing the number of missed diagnoses of early-stage feline hypertrophic cardiomyopathy prior to a crisis. Additional potential benefits of this approach include training veterinarians to recognize patients with feline hypertrophic cardiomyopathy prior to the development of acute cardiomyopathy, promoting the value of including an N-terminal pro-B-type natriuretic peptide (NT-proBNP) test as part of the work-up of patients with increased risk for HCM, and highlighting the potential significance of nonspecific recurring clinical signs in recognizing the risk for feline hypertrophic cardiomyopathy.

Determining whether a cat has HCM is of particular importance when cats undergo procedures that require anesthesia, for example dental work. Many pet owners decline further diagnostics and accept some additional risk (the magnitude of which is largely unknown) regarding anesthesia. However, owners would be more likely to pursue additional diagnostics if the veterinarian could ascertain that the likelihood of significant heart disease is high, which would influence subsequent anesthetic protocol, or determine that cardiac medications may be needed to help mediate disease. Cardiac blood tests, such as the NT-proBNP test, offer significant value over general blood work and chemistry panels in understanding the risk of underlying heart disease and, therefore, may allow the veterinarian to present a much more persuasive case to the pet-owner when advocating further diagnostics.

To help regulate fluid balance within the circulatory system, the heart produces diuretic and natriuretic hormones called natriuretic peptides. One particularly important peptide is B-type natriuretic peptide (BNP) that is produced in the ventricular and atrial myocardium. BNP is produced in response to stretch, hypoxia, and activity of other neuroendocrine pathways, such as the sympathetic nervous system. BNP is produced as a precursor molecule that is subsequently and enzymatically cleaved into the active neurohormone form of BNP and a by-product called N-terminal pro-B-type natriuretic peptide (NT-proBNP). NT-proBNP can be detected in the blood using standard laboratory techniques, such as an enzyme-linked immunosorbent assay, and its concentration reflects the degree of cardiac activation secondary to the above-discussed stimuli. The NT-proBNP test can also be used in cats with respiratory clinical signs to help differentiate cardiac disease versus primary respiratory etiologies (eg, asthma, pneumonia). In cats with respiratory signs, a low NT-proBNP concentration is highly specific as a rule-out test for congestive heart failure. A high NT-proBNP concentration is more consistent with congestive heart failure; however, in cats with both respiratory disease and concurrent mild or moderate heart disease, false positive results can occur. An NT-proBNP assay is not the gold standard for diagnosis of heart disease; echocardiography remains the diagnostic method of choice. However, while the NT-proBNP assay does not necessarily diagnose heart disease, it provides clinically significant guidance regarding the importance of further diagnostics, especially when asking the pet owner to pursue additional testing, such as echocardiography, radiography, and electrocardiography (ECG).

ECG is the gold standard for assessment of arrhythmias (and the least expensive of the diagnostics mentioned), but is relatively insensitive for detection of heart enlargement and dysfunction. Thus, many cats with underlying cardiomyopathy will have a normal ECG. If arrhythmias are detected or ECG criteria for left ventricular enlargement are met (for example, increased R-wave amplitude), the likelihood of underlying cardiac disease increases. Chest radiographs are a useful modality for evaluation of heart size and shape, and the ventrodorsal or dorsoventral view is the most sensitive for detection of feline atrial enlargement. However, the classic “valentine” shaped heart, which is highly specific for disease, is the exception rather than the rule in cats with mild or moderate asymptomatic disease. In instances when congestive heart failure (CHF) is suspected, chest radiographs are the gold standard for diagnosis of pulmonary edema or pleural effusion, but in cats with asymptomatic disease, clinical signs are by definition, absent, and CHF would not be expected. Thus, while a better diagnostic choice compared with ECG, radiography still suffers from relatively low sensitivity. Echocardiography is the diagnostic test of choice for detection of occult heart disease in cats. 2D and M-mode echocardiography provides detailed examination of ventricular and atrial dimensions, morphology, and function. Doppler echocardiography examines blood flow and detects abnormal mitral valve motion, mitral regurgitation, and high-velocity blood flow within the right ventricle, left ventricular outflow tract, and/or aorta, all of which are common causes of feline heart murmurs.

Although there is a marked lack of clinical indicators for feline HCM, there are a few biomarkers and plasma concentration tests that may indicate an increased risk for feline HCM. Evidence of hypercoagulability (increased likelihood of blood clotting) has been observed in about half of cats with HCM. Hypercoagulability is determined by comparing plasma concentrations of thrombin-antithrombin complex (TAT), D-dimer, and fibrin degradation products (FDP) in clinically healthy cats versus cats with HCM. Additionally, antithrombin activity has been observed to be significantly decreased in cats with HCM, and these markers may be useful in assessing risk for HCM. Another test that may be useful is the plasma concentration of cardiac troponin I (cTnI). Cardiac troponin I is attached to the actin filament of cardiac muscle and plays a role in cardiac myocyte contraction. Injury to cardiac myocytes causes release of cTnI, increasing the concentration of cTnI in the blood. Therefore, an increase in cTnI concentration may suggest heart damage, and it can be a good quantitative measure of the extent of heart damage; even though it does not provide any insight as to the cause of the damage. The cTnI concentration has been found to be both highly sensitive and highly specific for differentiating clinically healthy cats from cats with HCM, and has been found to aid in examining the severity of HCM and predict the development of further problems like heart failure. Although age and sex do not appear to have an effect on cTnI concentration in cats, body weight appears to affect cTnI concentration in healthy cats, possibly because an increased body weight may be associated with ventricular hypertrophy, which may cause an increase in the concentration of cTnI. Additionally, increased cTnI concentration has been correlated with other feline diseases, including kidney disease and hyperthyroidism. Therefore, it is important to consider the possibility of these diseases when using cTnI concentrations as a factor in the model for assessing the risk for HCM.

Hyperthyroidism is a fairly common disease in cats. Many typical clinical signs of cardiac disease are also often present in cats with hyperthyroidism, including tachycardia, tachypnea, and heart murmurs. As a result, many of the cardiac biomarkers and indicators of HCM may also be present in cats with hyperthyroidism, so additional tests may be required to rule out hyperthyroidism as a differential diagnosis. The most common screening performed for hyperthyroidism is simply a blood test, which measures the level of thyroid hormones T3 and T4, as well as the level of thyroid stimulating hormone (TSH). A positive indication of hyperthyroidism would be high levels of T3 and T4, with low levels of TSH. Using the T3/T4 levels as a factor in the machine learning model for feline HCM can help differentiate between hyperthyroidism and feline HCM. Additionally, sustained exposure to elevated levels of thyroid hormones may lead to cardiac hypertrophy, causing secondary HCM. Because hyperthyroidism is actually a major risk factor for developing HCM, it is quite difficult for a clinical to differentiate between a patient with hyperthyroidism that is causing other cardiac issues and mimicking common indicators for HCM, and a patient with hyperthyroidism that actually has developed HCM.

Other complications in properly identifying HCM include the possibility of cardiorenal syndrome and cardiohepatic syndrome. Cardiorenal syndrome refers to disorders of the heart and kidneys where dysfunction in one organ leads to dysfunction in the other. Although kidney disease is not known to cause feline HCM, kidney disease is known to cause other cardiac diseases that may influence the biomarkers associated with feline HCM. There are multiple biomarkers mainly associated with kidney disease, including symmetric dimethylarginine (SDMA) concentration, creatinine concentration, and Cystatin C (CysC) concentration, which can be used in the models to differentiate between primary HCM and cardiorenal syndrome. All of these markers are related to kidney function, specifically how well and how fast the kidneys are filtering blood. Although the effects of HCM are not entirely known on all of these biomarkers, studies have found that SDMA concentrations are generally not significantly different in cats with HCM and healthy controls, indicating that these biomarkers may be an extremely important tool in differentiating HCM from cardiorenal syndrome.

Cardiohepatic syndrome is similar to cardiorenal syndrome, referring to disorders of the heart and liver where dysfunction in one organ leads to dysfunction in the other. However, in contrast to cardiorenal syndrome, studies have found that many cats with HCM also show a slight increase in the liver enzymes alanine transaminase (ALT) and aspartate transaminase (AST). ALT is an enzyme that converts proteins into energy for liver cells, and AST is an enzyme that helps the body break down amino acids. Both ALT and AST are typically present in the blood at low levels, but are known to increase in concentration in response to liver damage. Additionally, AST activity has been known to increase in other cardiac and non-cardiac diseases, making it difficult to assess as a marker for HCM. These relationships are much less defined than those of cardiorenal syndrome, as no study has yet supported the presence of cardiohepatic syndrome in cats, further complicating the risk analysis of HCM in cats with known liver problems.

For the reasons discussed above, generating machine learning models to assess the risk of feline hypertrophic cardiomyopathy (HCM) poses several challenges. Conventional machine learning models work best when there is a large amount of training data with uniform distribution of positive and negative examples available, and the patterns that suggest the presence (positive) or absence (negative) of a particular disease are well-defined and separable from each other in the feature space. Although HCM is relatively common in felines, it is still much more likely to be negative than positive. HCM also has a wide variety of presentations from largely asymptomatic to severe complications. Thus, the training set for the diagnostic model for feline HCM is skewed, with fewer examples of positive data samples then negative data samples. Moreover, the patterns (in feature space) for positive data samples (from patients with feline HCM) are not well-defined and vary greatly, which makes it difficult to separate the clusters of positive samples from the clusters of negative samples (intermingling of positive and negative samples in the feature space) when training the diagnostic models.

To address these difficulties, a multi-stage approach is used to generate the diagnostic models 320. In some embodiments, the diagnostic models 320 for assessing the risk for feline HCM are generated using a supervised machine learning method. The supervised method involves utilizing very large sets of anonymized patient data to provide a labeled training set of defined positive cases (patients who have HCM) and negative cases (patients without HCM). In some embodiments, criteria for inclusion or exclusion of data samples in the training data set, and the definitions of positive and negative data samples, are defined by human medical experts. The computing device system 200, when presented with the training set, looks backwards at patient data samples in the months prior to the diagnosis of feline hypertrophic cardiomyopathy. From this data, the computing device system 200 uses mathematical algorithms to learn the patterns and relationships of weighted analytes and trends, along with other patient data, such as signalment, until one or more diagnostic models 320 that best predict the eventual diagnosis of feline hypertrophic cardiomyopathy are generated. Once the diagnostic models 320 are generated, they go through a preclinical technical validation for performance requirements on labeled data followed by a clinical evaluation in the field on unlabeled patient results before deployment.

In some embodiments, a training data set with a wide patient population showing diverse clinical presentations is collected. This permits the diagnostic models 320 to encode a more realistic representation of positive cases seen in general practice. The collected data set is filtered to remove data samples that represent outliers or secondary cases (with hyperthyroidism or hypertension induced HCM) that could improperly skew the diagnostic models 320. For example, data samples from patients who are already taking medications for HCM are removed from the training set, to ensure that the training set represents “natural” presentations of HCM. Similarly, data samples obtained from patients after a positive diagnosis for feline hypertrophic cardiomyopathy, or after a positive NT-proBNP test, may be removed because the patent may have been administered treatment that masks the natural indicators of HCM after positive diagnosis or based on the result of NT-proBNP test.

In some embodiments, the data preparation module 305 communicates with the one or more databases 330, 340, 350 to receive laboratory test data, and medical record and medical history data for a large population of patients, gathered over a period of time. In some embodiments, the medical record includes biographical information, such as age, gender, breed, and geographic location, one or more of which may be used as input features in training the machine learning models. These features can provide contextual discriminatory power to the machine learning models. In addition to biographical information, the medical record and the medical history data include information on any diagnostic tests that have been performed, and patient notes entered by a veterinarian. For example, a complete blood count (CBC) test includes data (results) for red blood cell count, hematocrit, hemoglobin, mean cell volume, mean corpuscular hemoglobin, mean corpuscular hemoglobin concentration, red blood cell redistribution width, reticulocytes (percentage and number), reticulocyte hemoglobin, nucleated red blood cells, white blood cell count, neutrophils (percentage and number), lymphocytes (percentage and number), monocytes (percentage and number), eosinophils (percentage and number), basophils (percentage and number), band neutrophils, platelet count, platelet distribution width, mean platelet volume, plateletcrit, total nucleated cell count, agranulocytes (percentage and number), and granulocytes (percentage and number). Other tests may check for presence, count, or concentration of squamous epithelial cells, non squamous epithelial cells, bacteria (rods or cocci), hyaline and nonhyaline casts, or crystals (bilirubin, ammonium biurate, struvite etc.). Levels of various proteins, enzymes, or minerals may have been checked. Other tests to check for specific pathogens may have been run, and their results stored in the medical record and the medical history data. Information on the diagnosis of feline hypertrophic cardiomyopathy, and the date of the diagnosis may also be stored in the medical record and medical history data.

In some embodiments, the data preparation module 305 receives the laboratory test data and the medical record and medical history data, and extracts features to be used as inputs for training machine learning models to classify feline hypertrophic cardiomyopathy. Ground truth (labeling of the data samples as positive or negative examples of feline hypertrophic cardiomyopathy) may be obtained from the diagnosis information, if expressly stored in the medical record and medical history data, or by using other criteria for labeling the data samples in the training data set. For example, in some embodiments, positive feline hypertrophic cardiomyopathy cases may be defined as data samples from patients with NT-proBNP results consistent with feline hypertrophic cardiomyopathy (for example, NT-proBNP concentration>100 pmol/L performed prior to administration of treatment for HCM) or an echocardiogram showing 6 mm of thickness in left ventricular wall. The remaining data samples, which do not meet the criteria for positive data samples, are labeled as negative examples. The data preparation module 305 also filters the collected data samples based on predefined exclusion criteria to generate a labeled training dataset.

In some embodiments, the data preparation module 305 determines the ratio of positive and negative labeled data samples in the training dataset and uses data sampling techniques to achieve a more balanced training dataset. For example, if the training data set has 10 times as many negative labeled examples as positive labeled examples, the data preparation module 305 may sample the negative examples to select 1 out of every 5 negative examples, to achieve a dataset that has twice as many negative labeled data samples as positive labeled data samples. It is readily understood that any predefined ratio for negatively labeled data samples to positively labeled data samples may be used, and the above example is provided merely for purposes of illustration only.

Data sampling techniques include random sampling, stratified sampling, cluster sampling, importance sampling, uncertainty sampling, and active learning, as examples. Random sampling entails randomly selecting the desired number of data samples from the larger pool of data samples. Random sampling is simple and cost effective, generally ensures that the selected subset is a representative sample of the overall data distribution, and can be implemented by randomly selecting a fixed number of instances or by specifying a percentage of the collected data samples to be included in the training set. Stratified sampling is useful when the class distribution of the selected data samples needs to be maintained. For example, in a case where the larger pool of collected data samples (negative examples) can be divided into different classes or categories, each class or category is proportionally represented in the selected data samples to ensure diversity of the data. Cluster sampling involves partitioning the data into clusters based on certain criteria (e.g., geographic location, demographics, or other relevant factors). Instead of sampling individual instances of patient data samples, each cluster is samples to ensure that a representative data set is collected. This can be beneficial when clusters represent distinct groups or subpopulations within the data, such as different breeds of animals. Importance sampling assigns weights to each data sample based on its importance or relevance to the diagnostic task. Data samples that are more informative or challenging can be given higher weights, while less informative samples receive lower weights. This technique allows prioritizing the inclusion of important data samples in the training dataset. Uncertainty sampling focuses on selecting data samples that the current model is uncertain about or finds challenging to classify. By selecting these data samples, the diagnostic system actively targets areas of the data where the trained machine learning model needs further improvement. Common uncertainty sampling strategies include selecting instances with the highest prediction entropy, margin, or confidence scores. Active learning is an iterative sampling approach where the trained machine learning model interacts with the data preparation module 305 to select the most informative data samples for further training. The model can query the diagnostic system for instances it is uncertain about or instances that are expected to have the most impact on improving its performance. This interactive process helps optimize the data selection based on the evolving needs of the machine learning models.

In some embodiments, the data samples in the training set are represented using feature vectors that include various analytes such as electrolyte levels (sodium, potassium, chloride, sodium-to-potassium ratio), glucose, urea (blood urea nitrogen-BUN), creatinine, albumin, alkaline phosphatase (ALP), red blood cell count (RBC), monocytes, lymphocytes, & lymphocytes reticulocyte, % reticulocyte, neutrophils, % neutrophils, eosinophils, % eosinophils, age, cholesterol, amylase, and calcium. It should be understood that the features included in the data samples are not limited to those listed here, and this list is provided for purposes of illustration only. Other features could be obtained from the patient's medical record and medical history or various laboratory tests such as CBC, basic chemistry panels, or more extensive chemistry panels, etc.

The feature space for assessing the risk of feline hypertrophic cardiomyopathy is very complex because of the potential number of features and because not all patients will have available information for all features. Complex feature spaces pose challenges for machine learning for several reasons such as dimensionality, computational complexity, overfitting, sparsity etc. As the number of potential features (dimensions) that can be used for assessing the risk of feline hypertrophic cardiomyopathy increases, the volume of the feature space grows exponentially. This phenomenon is known as the curse of dimensionality. In high-dimensional spaces, the available data becomes sparse, making it challenging for machine learning algorithms to generalize well. Models may struggle to find patterns or relationships when the number of features is much larger than the number of samples.

With an increasing number of features, the computational cost of training and evaluating machine learning models tends to rise. Many algorithms have time and space complexity that scales with the number of dimensions, making them computationally expensive in high-dimensional spaces. High-dimensional spaces also increase the risk of overfitting, where models capture noise or specific characteristics of the training data that do not generalize well to new, unseen data. This is also a direct consequence of the sparsity of data in complex feature spaces. Models may fit the training data too closely, capturing spurious correlations or outliers, rather than learning the underlying patterns because the available data points may be sparsely distributed. This sparsity makes it difficult for machine learning models to identify meaningful relationships between features and the target variable. Insufficient (sparse) data can also lead to poor model performance and unreliable predictions.

Some machine learning algorithms, especially those with a large number of tunable parameters, may become more difficult to train and fine-tune in high-dimensional spaces. This can lead to challenges in finding the right machine learning models for use in assessing the risk of feline hypertrophic cardiomyopathy. Moreover, some algorithms may become numerically unstable in high-dimensional spaces, leading to issues such as a lack of convergence during training or difficulties in optimizing the model parameters.

In some embodiments, the complexity of the feature space is reduced by performing feature selection—identifying only the most relevant/discriminative features and removing other features that do not significantly boost the performance of the machine learning models. However, it can be difficult to distinguish between informative and irrelevant features, leading to suboptimal model performance. To address these challenges, feature engineering, dimensionality reduction techniques (e.g., principal component analysis), and careful model selection are crucial. Ensemble learning, which combines multiple models, can be effective in mitigating the impact of complex feature spaces by leveraging the strengths of different models.

In some embodiments, ensemble learning is used to generate a plurality of machine learning diagnostic models 320 (ensemble of models 320), which are trained using the same or different sets of features. This approach permits the diagnostic system 300 to optimize different diagnostic models 320 on different portions of the sample space, and is especially useful for assessing the risk of feline hypertrophic cardiomyopathy due to its varied presentations. The outputs from the various machine learning diagnostic models 320 are merged or combined using ensemble methods to produce a single model/output that encompasses the strengths and compensates for the weaknesses of each individual diagnostic model 320.

In some embodiments, the machine learning model is generated by performing data collection, data splitting, model selection, model training, and model validation. Data collection has been discussed in detail above and entails gathering a representative data set that is relevant to assessing the risk of feline hypertrophic cardiomyopathy. Since bias and inconsistencies within the data can significantly impact a diagnostic model's performance, thorough data cleaning and pre-processing are performed. This includes addressing missing values in the data samples, identifying and correcting inconsistencies, and transforming the data into a format suitable for the selected type of machine learning model. In some embodiments, feature selection is performed to identify the most discriminative features and, potentially, reduce the complexity of the machine learning model. Once the data set is ready, it is divided into at least two separate training and test sets. In some cases, a separate validation set may also be generated. The training set serves as the labeled ground truth for training the diagnostic models 320 to learn the underlying patterns and relationships within the data. The test set is not presented to the models during training and is used to assess the models'ability to generalize and predict accurately on new data. The validation set is used during training to help the models generalize and avoid overfitting. During training, the machine learning algorithm iteratively adjusts the internal parameters of each diagnostic model 320 to minimize prediction errors on the training set. This process, known as optimization, aims to ensure that the diagnostic models 320 learn the optimal representations and decision boundaries for accurately classifying the data samples. The choice of appropriate hyperparameters, which control the learning process, plays a crucial role in achieving optimal performance. Hyperparameters govern the learning process and significantly impact the diagnostic model's performance. Tuning these parameters involves finding the optimal settings that maximize model accuracy and generalization. In some embodiments, techniques like grid search and random search are employed to systematically explore the parameter space and identify the optimal configuration. Once the diagnostic models 320 are trained, their performance is evaluated using the test set. This involves measuring the models' accuracy, precision, recall, and other relevant metrics. These metrics provide valuable insights into the models'strengths and weaknesses, allowing for further refinement and improvement. Cross-validation, which involves evaluating the models on multiple splits of the data, provides a more robust estimate of their generalization ability. Both hyperparameter tuning and cross validation can be performed during either of the training or the testing phases, to improve the models'performance.

Ensemble learning is an advanced machine learning technique that combines predictions from multiple diagnostic models 320 to produce a more accurate and robust final prediction. This approach leverages the diversity of individual diagnostic models 320 to mitigate biases and further improve overall performance, making it particularly effective in complex and varied datasets. The idea behind ensemble learning is that by combining diverse independent models, the weaknesses of one model can be compensated for by the strengths of others, and the aggregation of the models result in a more accurate and reliable prediction. Diversity means that the different machine learning models within the ensemble make different types of errors. This diversity ensures that the ensemble of models 320 can collectively address a broader range of scenarios. The models within the ensemble are designed to be as independent as possible to reduce the risk of systematic errors, because similar models might make the same mistakes. The predictions of individual models 320 are combined through a predefined aggregation strategy, such as averaging, voting, or weighted voting. The goal is to leverage the collective intelligence of the ensemble.

The machine learning models in the ensemble of models 320 can be homogeneous, heterogeneous, or a mix of both. Homogeneous models refer to using more than one machine learning model of the same type, but with intentional variations. For instance, in the case of a decision tree machine learning model, different decision trees having different depths, minimum samples per leaf, or feature subsets maybe generated during training. Heterogeneous models involve combining different types of models, each contributing unique perspectives. This could include combining decision trees with support vector machines, k-nearest neighbors, or neural networks, creating a diverse set of predictors.

In some embodiments, one or more ensemble learning training methods are used to train the ensemble of diagnostic models 320. The ensemble learning training methods include bagging, boosting, and stacking. Bagging, also known as bootstrap aggregating, involves generating multiple instances of a same type of machine learning model on different subsets of the training data. Each subset is obtained through bootstrapping (random sampling with replacement or another data sampling technique discussed above). Each instance of the machine learning model is trained independently using one subset of the training data. This process introduces diversity by exposing each model to slightly different instances and reduces overfitting. Different operators, such as the average (for regression) or majority vote (for classification) of the individual model predictions, are used for the final prediction. In some embodiments,

Boosting entails building a sequence of diagnostic models, where each subsequent diagnostic model in the sequence tries to reduce the errors of the preceding diagnostic model in the sequence. Boosting assigns weights to instances based on their performance in earlier iterations, emphasizing the importance of misclassified data points. Higher weights are assigned to misclassified instances and lower weights to correctly classified ones and a weak learner is trained on the weighted dataset. The model's performance is evaluated using the test data set and the errors are used to adjust weights. This process is repeated iteratively, giving more emphasis to misclassified instances in each iteration, until the error in prediction is below a desired threshold. Algorithms like AdaBoost and Gradient Boosting exemplify this approach, refining the model iteratively to enhance overall predictive performance.

Stacking involves training multiple different types of machine learning models using the techniques discussed above, and then combining their predictions using another model called a meta-model or blender. The meta-model learns to weigh the predictions of the individual models to create a final prediction.

To ensure robustness and generalization of the trained diagnostic models 320 in the ensemble, both technical (cross-fold) and real-world (clinical trial) validation is employed. This rigorous validation process helps prevent overfitting and provides a more accurate estimate of the models' performance. The predictions from each of the plurality of diagnostic models in the ensemble maybe combined using different functions to generate a single final prediction. For example, in hard voting, the final prediction is determined by the majority vote of the diagnostic models 320. Each diagnostic model has equal weight in the decision-making process. As another example, soft voting considers the confidence or probability assigned to each class by each diagnostic model 320. The final prediction is a weighted combination of these probabilities, providing a more nuanced decision. As yet another example, weighted averaging may be used to assign a weight to each model's prediction based on the model's performance during validation (technical and/or clinical trial). In weighted averaging, diagnostic models with higher accuracy or reliability are given more significant influence in the final prediction, allowing for a dynamic weighting scheme.

FIG. 4 shows a flowchart of an ensemble learning based feline hypertrophic cardiomyopathy risk assessment method 400, according to various embodiments. In step 410, the data preparation module 305 prepares the data sets for training, testing, and validating the diagnostic models 320 for the diagnostic system 300. The raw data samples are obtained from diagnostic tests performed either at laboratories or using diagnostic instruments at the POC terminal, and clinical history data derived from integrated veterinary clinic practice information management software (PIMS). In some embodiments, the data samples are collected over a period of time and stored in the one or more databases 330, 340, and 350, discussed above. In some embodiments, ground truth for the data samples is defined as NT-proBNP concentration>100 pmol/L, consistent with feline hypertrophic cardiomyopathy. A wide patient population with diverse clinical presentations is included in the training data set to provide the diagnostic models 320 with a more realistic representation of the general patient population. Since the goal of the diagnostic system 300 is to detect early or preclinical cases, the positively labeled data samples are not restricted to those with clinical signs or to patients in whom feline hypertrophic cardiomyopathy is already suspected. In some embodiments, medical experts defined an HCM phenotype to label positive data samples (i.e., patients with feline hypertrophic cardiomyopathy) and negative data samples (i.e., patients without feline hypertrophic cardiomyopathy). The phenotype involves several inclusion and exclusion criteria to label data samples as positive or negative. For example, positive data samples include patients with NT-proBNP test results consistent with increased risk of HCM (for example, NT-proBNP concentration>100 pmol/L). In some embodiments, the collected data samples are divided into a plurality of data subsets for training, validating, and testing the diagnostic models.

The data samples in the training, validating, and testing data sets include features such as complete blood count and serum chemistries (e.g., blood urea nitrogen [BUN], sodium, and potassium) as well as demographic data like gender, breed, and age. The minimum requirements for the diagnostic models include commonly assessed parameters as found in CBC and basic chemistry panels with electrolytes.

In step 420, the data set is filtered by one or more criteria discussed above to extract the relevant set of data samples for training, validating, and testing the machine learning models 320. In step 430, the diagnostic model training module 310 selects and trains an ensemble of diagnostic models 320 using the ensemble learning methods discussed previously. In some embodiments, in step 430, a plurality of decision trees (random forest of trees) is trained on different feature subsets in the data samples to generate the ensemble of diagnostic models 320. A decision tree is a machine learning model with a tree-like structure where each internal node represents a feature (or attribute) of the data set, and each branch represents a possible outcome of the feature. At each node, the algorithm selects the best feature based on a splitting criterion, such as information gain or Gini impurity. This process continues recursively until reaching a leaf node, which represents the final predicted class or value. Decision trees are easy to interpret and understand, and can be generated using expert domain knowledge (a rule based approach). The tree structure provides a clear visual representation of the decision-making process. Moreover, decisions trees are robust to irrelevant features because the algorithm automatically ignores features that are not relevant for prediction. However, decision trees are prone to overfitting and can become too complex in a large feature space, leading to poor performance on unseen data. Decision trees are also sensitive to noise in the data; slight changes in the data can lead to significant changes in the tree structure. Each tree in a random forest is trained independently and makes its own prediction. The final prediction is then obtained by taking the majority vote from all trees. This approach is more robust to outliers and noise in the data.

Random forests are ensemble learning algorithms that combine multiple decision trees to improve overall performance. They work by building a forest of individual decision trees, each trained on a different subset of the data and using a random subset of features at each split. The predictions from these individual trees are then combined to make the final prediction. Random forests provide improved accuracy and robustness over individual decision trees. By combining multiple decision trees, random forests are less prone to overfitting and more robust to noise in the data, and can handle complex relationships between features. Random forests can learn complex relationships between features that may not be easily captured by a single decision tree. Although more computationally expensive to train, and less interpretable than individual decision trees, the random forest ensemble models outperform individual decision trees because assessing the risk of feline hypertrophic cardiomyopathy is a complex problem with many features and intricate relationships.

In some embodiments, in step 430, a gradient boosting approach is used to build a sequence of decision trees using ensemble learning. Gradient boosting is a powerful technique for building ensemble models by sequentially adding weak machine learning models (models with low individual accuracy) to improve overall performance. The training process starts with an initial model, often a simple model like a single decision tree. The algorithm calculates the pseudo-residuals, which represent the difference between the actual and predicted values for each data sample, and iteratively adds weak machine learning models. In each iteration, a new weak machine learning model is trained on the current pseudo-residuals. This new machine learning model focuses on correcting the errors made by the previous machine learning models. The learning rate determines the weight given to each new machine learning model in the ensemble. The (weighted) predictions of all weak machine learning models are combined to generate the final prediction at each iteration. The iteratively process of adding weak machine learning models and updating predictions continues until a stopping criterion is met. Common stopping criteria include reaching a desired level of accuracy or exceeding a maximum number of iterations. Some benefits of using gradient boosting to build an ensemble model include improved accuracy, reduced variance, flexibility, and scalability. By combining the predictions of multiple weak machine learning models, gradient boosting can achieve significantly higher accuracy than individual learners. Predictions are made by majority vote of the weak machine learning models; predictions, weighted by their individual accuracy. Further, since gradient boosting focuses on correcting errors made by previous machine learning models, leading to a more robust ensemble model with lower variance. Gradient boosting can be used with various types of machine learning models, such as decision trees, linear regression models, and others, making it easy to generate heterogenous models for the ensemble of diagnostic models.

In steps 440 and 450, the diagnostic model selection module 315 performs technical and clinical validation of the trained ensemble of diagnostic models 320, respectively. Technical validation, performed in step 440, ensures that the diagnostic models 320 meet the performance requirements, are unbiased, and are strong enough to support a clinical evaluation prior to deployment. The preclinical technical validation is performed by running the model against the validation and test data sets of cases. In some embodiments, the training set is used to train the ensemble model, the validation set is used to tune hyperparameters of the ensemble model and monitor the model for overfitting during training. The test set is used to evaluate the final model's performance on unseen data. The machine learning models used in ensemble learning (whether random forest, gradient boosting models, or other machine learning models) have several hyperparameters that can significantly impact their performance. Techniques like grid search or random search can be used to identify the optimal values of these hyperparameters. The validation data set is essential for evaluating the performance of different hyperparameter configurations and selecting the best ones for generating the structure of the machine learning models included in the ensemble. In some embodiments, metrics such as loss function, accuracy, and AUC can be monitored during model training and validation to improve the model's performance.

In some embodiments, a loss function measures the difference between the predicted and actual values for a data sample. Lower values of the loss function indicate better model performance. Common loss functions include mean squared error (MSE), which measures the average squared difference between predicted and actual values, cross-entropy, which measures the difference between the predicted probability and the actual class, and logarithmic loss, which is like cross-entropy but more sensitive to misclassified examples. Accuracy represents the percentage of predictions that are correct. It is a simple and intuitive metric but can be misleading when dealing with imbalanced datasets. AUC (area under the curve) measures the ability of a classifier to distinguish between classes. The AUC represents the area under the receiver operating characteristic (ROC) curve, which plots the true positive rate (TPR) against the false positive rate (FPR) at different thresholds. An AUC of 1 represents a perfect classifier, while an AUC of 0.5 represents a random guess. In some embodiments, one or more of the loss function, accuracy, and AUC are observed. If these metrics plateau or worsen on the validation data set, the trained ensemble model likely suffers from overfitting. In this case, early stopping techniques are used to stop training once the validation metrics start declining, preventing overfitting.

In some embodiments, cross validation is used to monitor the ensemble diagnostic model 320 for overfitting. Cross-validation is a resampling procedure used to evaluate machine learning models on a limited data sample. The procedure has a single parameter called k that refers to the number of groups that a given data set is to be split into. As such, the procedure is often called k-fold cross-validation. When a specific value for k is chosen, such as k=10, the procedure becomes 10-fold cross-validation. Cross-validation is primarily used to estimate how the trained model is expected to perform in general when used to make predictions on data not used during the training of the models 320. The dataset is shuffled randomly and divided into a predefined number (k) of groups. The training and validation process is performed k times, with one of the groups of data being held out as the validation set for each iteration and the remaining k−1 groups being used as the training set. Each model is fitted (trained) on the training set and evaluated (validated) on the test set to determine the level of generalization of the trained models. The purpose of k-fold cross validation is not to pick one of the trained models 320 as the final machine learning model but, rather, to help determine the model structure and the hyperparameter tuning process for each machine learning model 320. Once the ensemble model 320 is trained and hyperparameters are tuned, its performance is evaluated using the test data set.

In step 450, a clinical validation of the ensemble of diagnostic models 320 is performed to measure the model's effectiveness in assessing the risk for feline hypertrophic cardiomyopathy, prior to deployment (step s460). In some embodiments, the clinical validation is performed by deploying the diagnostic models 320 on a test basis at a selected number of clinics, evaluating new data samples from patients using the trained diagnostic models 320, and validating the prediction of the diagnostic models 320 using expert opinion of the veterinarians. Further diagnostic tests may be run, in the case where the ensemble model 320 predicts a higher risk of feline hypertrophic cardiomyopathy, to determine whether the patient is suffering from feline hypertrophic cardiomyopathy.

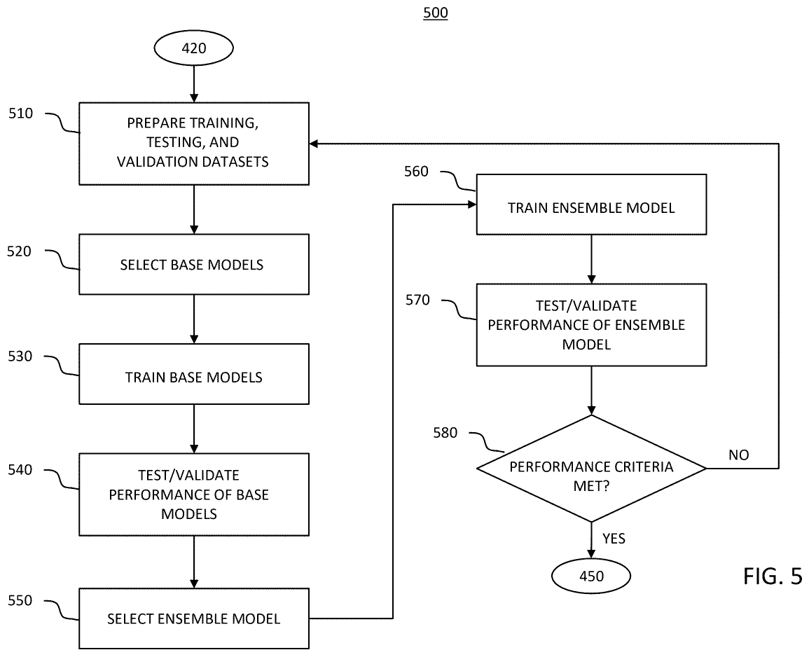

FIG. 5 shows a flowchart with additional details for a method 500 of training the ensemble of diagnostic models 320, according to some embodiments. In step 510, the collected and filtered medical data (from steps 410 and 420) is pre-processed and split into training, testing, and validation datasets using the sampling techniques discussed above. In step 520, the individual base models (learners) for the ensemble of models are selected. The learners may include one or more machine learning models such as decision trees, random forests, support vector machines, neural networks etc. A diverse set of learners may be selected for better ensemble performance. Multiple instances of a same type of machine learning model with different architecture or parameter sets may also be selected. In step 530, each base model (learner) is trained independently on the training data set using model-specific training methods. In step 540, each base model is tested/validated for performance using technical validation methods such as cross-validation. The training and validating steps 530 and 540 are repeated iteratively until predefined performance criteria for each base model is met. Performance metrics (e.g., accuracy, loss) on training and testing/validation data are measured and tracked to improve the performance of each base model. In some embodiments, early stopping is used to prevent overfitting during the training step 530 based on the performance metrics. During the training and validating steps 530 and 540, the hyperparameters (e.g., tree depth, number of estimators etc.) for each model are tuned.

In step 550, the ensemble architecture (e.g., bagging, boosting, stacking), the learning approach (e.g., parallel, sequential), and the prediction aggregation strategy (e.g. averaging, voting) is selected. In step 560, the ensemble of diagnostic models 320 is trained using the outputs or predictions of the base models. In some embodiments, the base models may be weighted differently based on their performance, permitting models with better performance to have more influence on the output of the ensemble model 320. In step 570, the ensemble model's performance is validated on unseen validation data. The ensemble model's performance is also compared with individual base model performance to check for overfitting (high training accuracy, low validation accuracy). In some embodiments, in step 580, results from the ensemble model 320 are analyzed. If the performance meets the desired criteria, the ensemble model is ready for clinical validation and deployment. If the performance does not meet the desired criteria, areas for improvement are identified and steps 510-570 are repeated to adjust the base models, ensemble architecture, hyperparameters, or data pre-processing as needed. Training and validation of the individual learners and the ensemble model are repeated until the desired performance is achieved.

In some embodiments, the diagnostic models 320 (the ensemble of diagnostic models) include at least a first machine learning diagnostic model that is trained on electrolyte abnormalities/features and a second machine learning diagnostic model that is not trained on electrolyte abnormalities/features. In some embodiments, the diagnostic system 300 includes a graphical user interface 360 that permits a user to further engage with an interactive diagnostic model 370 when any of the diagnostic models in the ensemble of models 320 indicate a risk of feline hypertrophic cardiomyopathy.

In some embodiments, the diagnostic models 320 are implemented as artificial neural networks (ANNs). The artificial neural network comprises a plurality of interconnected artificial neurons, where each artificial neuron receives input signals, applies weighting functions, and generates output activations based on predetermined activation functions. A multi-core CPU architecture of a data processing device system 110 physically implements these artificial neurons through coordinated processing operations across multiple processing cores, with each core capable of executing computations for multiple artificial neurons simultaneously. The memory device system 130 stores synaptic weight values that define the connections between artificial neurons, with optimized data structures that enable rapid retrieval of weight parameters during neuron activation computations. This hardware-to-neuron mapping provides a direct correspondence between the physical computing resources and the logical structure of the artificial neural network, enabling efficient execution of neural network operations on a computing device 200.

In some embodiments, the artificial neural networks are implemented on a system 100, including a data processing device system 110 and a memory device system 130, which are configured in a specific architecture to optimize neural network processing. The data processing device system 110 may include a central processing unit (CPU), having multiple processing cores that are each configured with specialized instruction sets for neural network operations. The CPU implements vectorized arithmetic operations and parallel execution units optimized for matrix computations commonly used in neural network processing. Each processing core includes dedicated registers for storing intermediate neural network computation results and specialized cache configurations to minimize memory access latency.

In some embodiments, the memory device system 130 comprises system RAM configured with optimized data structures for neural network weight storage and intermediate result caching. The memory architecture implements specific addressing schemes and data layout patterns that align with neural network computation requirements. The memory device system 130 utilizes memory hierarchies including multiple cache levels specifically configured for neural network data access patterns. Neural network weights are stored in the memory device system 130 using specific data structures optimized for rapid access during computation cycles. The system 100 includes weight quantization techniques that reduce memory requirements while maintaining computational accuracy. Weight updates during training operations utilize optimized memory write patterns that minimize cache invalidation and improve overall system performance.

In some embodiments, the data processing device system 110 includes parallel processing capabilities using multiple CPU cores and specialized processing units. Each processing unit executes specific portions of neural network computations simultaneously, with coordinated data sharing and synchronization mechanisms. The parallel architecture includes dedicated communication pathways between processing units to minimize data transfer overhead. Parallel processing coordination logic distributes neural network layer computations across the multiple processing cores with synchronized data sharing. The coordination logic implements load balancing algorithms that distribute neural network computations across available processing resources to maximize utilization efficiency.

The forward propagation process executes using a series of matrix multiplication operations distributed across multiple processing units. Each layer of the neural network executes on designated processing units of the data processing device system 110 with optimized data flow patterns. The system includes specific scheduling algorithms that coordinate execution across the multiple processing units to minimize idle time and maximize throughput. During forward propagation, input data flows through successive neural network layers, with each layer performing weighted sum computations followed by activation function applications. The weighted sum computations utilize optimized matrix multiplication algorithms that leverage the parallel processing capabilities of multiple CPU cores.

In some embodiments, activation functions for the neurons execute using specialized lookup tables and interpolation algorithms optimized for the specific processing capabilities of the standard computer hardware. The system 100 includes multiple activation function types including sigmoid, rectified linear unit (ReLU), and hyperbolic tangent (tanh) functions with hardware-optimized computation methods. For sigmoid activation functions, the system utilizes piecewise linear approximations stored in cache-optimized lookup tables. ReLU activation functions execute using conditional branching operations optimized for the branch prediction capabilities of the CPU architecture. Tanh activation functions implement Taylor series approximations with coefficients pre-computed and stored in dedicated cache memory. Training operations execute using distributed gradient computation across multiple processing units. The system 100 includes specific algorithms for gradient accumulation and weight update operations that utilize the parallel processing capabilities of the computing device 200. Error propagation calculations execute using optimized matrix operations with specialized memory access patterns. During backpropagation training, error gradients propagate backward through the neural network layers, computing partial derivatives with respect to each weight parameter. The gradient computation utilizes chain rule applications implemented through coordinated matrix operations across multiple processing cores.

In some embodiments, the system 100 includes cache optimization strategies that align neural network data access patterns with the cache hierarchy of the computing device 200. Instruction scheduling algorithms optimize the sequence of neural network operations to maximize utilization of parallel execution units within the CPU. Cache management includes predictive prefetching of neural network weights based on anticipated computation sequences. The cache hierarchy utilizes level 1 instruction and data caches dedicated to each processing core, shared level 2 caches for coordinated operations between cores, and level 3 cache memory for neural network weight storage and intermediate result buffering.

In some embodiments, memory bandwidth utilization optimization is performed using specific data prefetching strategies that anticipate neural network computation requirements and pre-load data into appropriate cache levels. The system 100 includes branch prediction optimizations for conditional operations within neural network computations. Data prefetching algorithms analyze neural network layer connectivity patterns to predict required weight parameters and activation values for subsequent computational cycles. The prefetching mechanism reduces memory access latency by maintaining frequently accessed neural network parameters in high-speed cache memory.

In some embodiments, the system 100 provides specific technical improvements to computer processing efficiency by implementing optimized algorithms that reduce computational complexity for neural network operations. The system 100 achieves reduced processing latency through specialized instruction sequencing and parallel execution coordination. Computational efficiency improvements include vectorized instruction execution that processes multiple neural network operations simultaneously within single CPU cycles. The vectorized operations utilize Single Instruction Multiple Data (SIMD) capabilities of modern CPU architectures to perform parallel arithmetic operations on neural network data arrays. Memory access patterns are optimized through specific addressing schemes and data structure layouts that align with the cache architecture of standard computer hardware. The system 100 includes prefetching strategies that reduce memory access latency and improve overall computational throughput. Neural network weight matrices utilize row-major or column-major storage layouts selected based on the specific access patterns of forward and backward propagation algorithms. The memory layout optimization ensures that sequentially accessed neural network parameters reside in contiguous memory locations to maximize cache line utilization.

The specialized architecture provides scalability improvements by implementing distributed processing methods that efficiently utilize multiple CPU cores and processing units. In multi-node configurations, the system 100 distributes neural network layers across multiple computing devices 200 (nodes) connected via high-bandwidth network interfaces. Each node executes specific neural network layers with coordinated data transfer for intermediate results. Network communication protocols optimize data transfer patterns to minimize latency between distributed processing nodes. Weight synchronization in distributed configurations utilizes parameter server architectures where dedicated nodes maintain authoritative copies of neural network weights. Processing nodes retrieve weight updates through optimized communication protocols that minimize network bandwidth utilization. Gradient aggregation across distributed nodes implements averaging algorithms that maintain numerical stability and convergence properties of the neural network training process.

In some embodiments, the system 100 include fault tolerance mechanisms for maintaining computation integrity across distributed processing configurations. Checkpoint operations periodically save neural network state information to persistent storage systems. In the event of processing node failures, the system 100 restores neural network computations from checkpoint data and redistributes processing loads across remaining functional nodes. In some embodiments, the system 100 includes power management systems that configure CPU frequency scaling and memory access patterns based on neural network computation requirements. During intensive matrix multiplication phases, the system 100 increases CPU frequency to maximize computational throughput. During memory-intensive operations such as weight loading, the system 100 optimizes memory bus utilization while reducing CPU frequency to minimize power consumption. The power management algorithms balance computational performance with energy efficiency based on real-time analysis of neural network processing workloads.

In some embodiments, the system 100 includes monitoring and profiling capabilities that analyze neural network execution patterns and identify performance optimization opportunities. Performance counters track cache hit ratios, memory bandwidth utilization, and parallel processing efficiency. The profiling data enables dynamic optimization of neural network processing parameters including thread allocation, cache management policies, and memory access patterns. Adaptive algorithms adjust system configurations based on observed performance characteristics to maximize computational efficiency for specific neural network architectures and datasets.

In these embodiments, the ensemble of diagnostic models 320, discussed above, provides an indication that the veterinarian should perform additional screening to assess the risk for feline hypertrophic cardiomyopathy. In some embodiments, the additional screening is performed in an interactive manner by a user (for example, the veterinarian) engaging with the interactive diagnostic model 370 via the user interface 360. The user interface presents a sequence of questions, dynamically selected by the third diagnostic model 370 based on answers provided to previous questions, to guide the veterinarian towards additional testing to confirm the presence or absence of feline hypertrophic cardiomyopathy.

For example, if either the first diagnostic model or the second diagnostic model in the ensemble of models 320 indicate that the patient may have feline hypertrophic cardiomyopathy, the third diagnostic model 370 may first query the veterinarian to provide additional information on recent medications taken by the patient. If the patient is not taking medications that cause the patient's bloodwork to have similar patterns as those for feline hypertrophic cardiomyopathy, then the third diagnostic model 370 may further query the veterinarian for additional information on clinical presentation and patient history. This information is utilized along with results of recent tests (such as NT-proBNP etc.) to train the third diagnostic model 370 to provide further diagnostic guidance and evaluation.

In some embodiments, the third diagnostic model 370 includes a knowledge based expert system that combines both contextual domain knowledge and data-driven training to improve diagnostic accuracy and efficiency. In some embodiments, the training phase of the third diagnostic model 370 includes generating a knowledge base that encodes expert knowledge in diagnosing feline hypertrophic cardiomyopathy. The expert knowledge is obtained by interviewing medical professionals specializing in feline hypertrophic cardiomyopathy to capture their diagnostic reasoning, differential diagnoses, and treatment strategies. The expert knowledge may also be gathered by analyzing (using either human experts or machine learning models) medical textbooks, research papers, documented cases (data) and guidelines for evidence-based practice. The acquired knowledge is appropriately structured and encoded for the training to be performed efficiently. For example, in some embodiments, the knowledge about relationships between symptoms, findings, and diagnoses can be captured using production rules such as “IF-THEN” statements. In other embodiments, the knowledge may be encoded as a Bayesian networks, which represents causal relationships between factors using probability. This permits the third diagnostic model 370 to consider the likelihood of different diagnoses based on various combinations of symptoms and test results. In yet other embodiments, a semantic network may be used to connect symptoms, diseases, and tests through directed links, creating a web of interconnected knowledge that facilitates efficient information retrieval and inference.

In some embodiments, the expert knowledge defines general rules and guidelines for the knowledge base, which are then refined using patient medical history and test data (such as NT-proBNP test, etc.) to train the third diagnostic model 370 to provide further diagnostic guidance and evaluation. The data is collected from diverse data sources, including electronic medical records, laboratory test results, clinical signs, and medication histories. The data is cleaned and validated, any missing or inconsistent values are addressed, and potential biases are removed before using the data for training. Data mining techniques like association rule mining and clustering are employed to identify hidden patterns and correlations within the data. This data mining analysis uncovers associations between specific symptom combinations and diagnoses, or reveal risk factors and disease progression patterns. The patterns and insights extracted using data mining are then used to refine the existing rules in the knowledge base. The data mining techniques used for training the third diagnostic model 370 automatically discover previously unknown relationships between symptoms and diagnoses, not readily apparent from expert knowledge alone. This allows the third diagnostic model 370 to stay relevant and adapt to evolving disease patterns and emerging medical literature.

In some embodiments, the third diagnostic model 370 integrates probabilistic reasoning techniques with a rules-based engine to account for data variability and incomplete information. This enables the diagnostic system 300 to provide confidence levels associated with diagnoses, enhancing transparency and guiding further investigative steps. The third diagnostic model 370 employs an inference engine that translates the knowledge base and the responses to the queries from the user interface 360 into actionable insights. In some embodiments, the inference engine includes both forward chaining and backward chaining inference strategies.

Forward chaining starts with known facts (symptoms, test results) and applies the rules iteratively to infer additional facts and ultimately reach a diagnosis/conclusion. Backward chaining works backward from a suspected diagnosis/conclusion, identifying supporting evidence and tests needed to confirm or refute it. Backwards chaining is especially useful to identify additional information that is needed to support a diagnosis or conclusion, and query the veterinarian, using the graphical user interface 360, to provide the additional information. The user interface 360 of the diagnostic system 300 permits the veterinarian to not only input patient symptoms but to also assign relative weights to their severity or duration, providing the diagnostic system 300 with richer context.

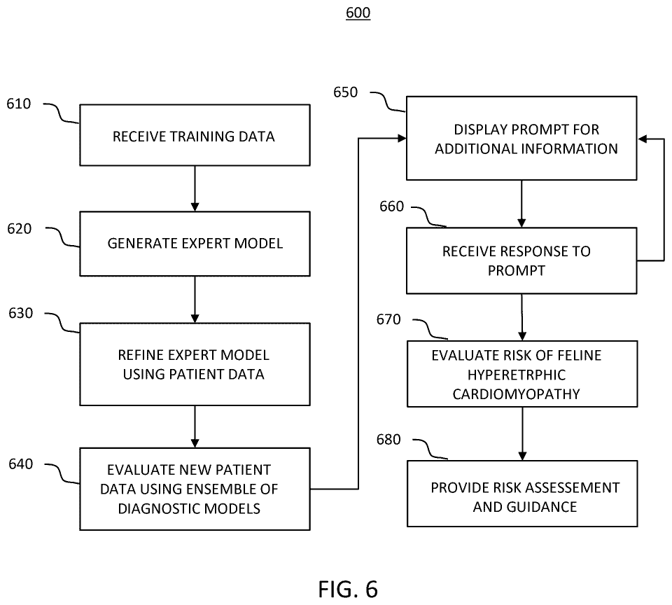

FIG. 6 shows a flowchart of a method 600 for training and using an interactive knowledge based model for assessing feline hypertrophic cardiomyopathy risk, according to various embodiments. In step 610, data for training the knowledge based model is obtained. The training data includes both domain knowledge and clinical/laboratory patient data. The domain knowledge is obtained, for example, from human experts. In step 620, an expert model is generated using the obtained domain knowledge. The expert model may include rules-based inference, knowledge graphs, semantic ontologies, predicate logic or other techniques, known in the art, for representing domain knowledge. In step 630, the expert model is refined using the clinical/laboratory patient data. This refinement could include adjusting the logic defined in the expert model, assigning weights to the various logic branches, adjusting the connections between various entities in the model etc.

In step 640, new patient data is received and assessed using the ensemble of diagnostic models 320. If the ensemble of diagnostic models indicates a potential risk of feline hypertrophic cardiomyopathy, in steps 650 and 660, an interactive user interface is used to present a clinician with a sequence of prompts and receive responses to the sequence of prompts, to acquire additional data to assess the risk of feline hypertrophic cardiomyopathy. Each new prompt presented in step 650 may be based on the response received to a previous prompt in step 660. Steps 650 and 660 may be repeated, as appropriate, to acquire the additional information required by the knowledge based model. In step 670, the risk of feline hypertrophic cardiomyopathy is determined based on the acquired additional data. In step 680, the user interface is used to provide the clinician with an assessment of the risk for feline hypertrophic cardiomyopathy on the new patient data and guidance for further testing and treatment.

In some embodiments, the diagnostic system 300 provides differential diagnosis suggestions, presenting a list of possible diagnoses based on the entered information, considering the patient's medical history and test results. In some embodiments, the diagnostic system 300 provides evidence-based recommendations, suggesting further tests or investigations based on the third diagnostic model's 370 assessment and current clinical guidelines. In some embodiments, the diagnostic system 300 provides explanatory reasoning for the diagnostic model's conclusions and guidance, showing the reasoning chain behind the suggested diagnoses and tests, promoting transparency and trust with the veterinarian.

In some embodiments, the diagnostic system 300, including the ensemble of diagnostic models 320 and the third diagnostic model 370, is continually evaluated and refined using anonymized patient data to assess its accuracy in clinical settings. Validation of the diagnostic system 300 is also performed based on feedback from medical professionals using the diagnostic system 300, to identify areas for improvement and ensure its practical utility. In some embodiments, the knowledge base is regularly updated to incorporate new medical knowledge from medical experts, research, guidelines updates, and emerging disease patterns.

In some embodiments, the diagnostic system 300 provides information on therapeutic methods and compositions for the treatment of feline hypertrophic cardiomyopathy. The therapeutic methods include administering pharmacological agents that target the specific pathophysiological mechanisms. For example, atenolol, a beta-adrenergic receptor antagonist, is administered orally at dosages of 6.25-12.5 mg once or twice daily, or 1-2 mg/kg once or twice daily. Atenolol reduces myocardial oxygen demand and improves diastolic filling by prolonging the diastolic filling period. Atenolol demonstrates particular efficacy in cases with dynamic left ventricular outflow tract obstruction by reducing the pressure gradient across the outflow tract. In some embodiments, a calcium channel blocker such as diltiazem is used as an alternative or adjunctive therapeutic approach, with oral dosages of 7.5-15 mg twice or thrice daily, or 1-2.5 mg/kg. The therapeutic benefit for calcium channel blockers derives from improved diastolic relaxation through interference with calcium-mediated excitation-contraction coupling, enhanced lusitropy, and modest negative inotropic effects that reduce outflow tract obstruction.

In some embodiments, angiotensin-converting enzyme (ACE) inhibitors are used for treatment in cases with congestive heart failure or as preventive therapy in asymptomatic feline patients with severe left atrial enlargement. For example, enalapril at 0.25-0.5 mg/kg once or twice daily, or benazepril at 0.25-0.5 mg/kg once daily, may provide afterload reduction and neurohormonal modulation. The mechanism involves inhibition of angiotensin II formation, reducing vasoconstriction, aldosterone secretion, and myocardial remodeling.

In some embodiments, diuretic therapy becomes necessary in cases presenting with congestive heart failure manifestations. Furosemide administered at initial dosages of 1-2 mg/kg twice daily, with titration based on clinical response and potential escalation to 2-4 mg/kg twice or three times daily in refractory cases, provides preload reduction through sodium and water elimination. The therapeutic effect involves decreased venous return, reduced left atrial pressure, and alleviation of pulmonary edema.

In some embodiments, anticoagulation therapy addresses the significant thromboembolic risk associated with feline HCM, particularly in feline patients with spontaneous echocardiographic contrast, left atrial enlargement exceeding 20 mm, or history of arterial thromboembolism. Clopidogrel at 18.75 mg once daily represents the current standard, functioning through irreversible inhibition of the P2Y12 adenosine diphosphate receptor on platelets, thereby preventing platelet aggregation and thrombus formation.

In some embodiments, a combination therapy is used to treat feline HCM. Combination therapy protocols involve strategic utilization of multiple pharmacological agents to address different pathophysiological components simultaneously. A typical regimen for a feline patient with HCM and mild congestive heart failure might comprise atenolol 6.25 mg twice daily combined with enalapril 1.25 mg once daily and furosemide 12.5 mg twice daily, with clopidogrel 18.75 mg once daily for thromboembolic prophylaxis. Monitoring protocols require serial evaluation of therapeutic response through echocardiographic assessment, including measurement of left atrial dimensions, left ventricular wall thickness, presence and degree of systolic anterior motion of the mitral valve, and peak left ventricular outflow tract velocity. Biomarker monitoring may include N-terminal pro-B-type natriuretic peptide (NT-proBNP) levels, with values exceeding 270 pmol/L indicating increased risk of cardiac events. Dosage modifications become necessary based on individual patient response, concurrent medications, and development of adverse effects. Renal function monitoring is essential when utilizing ACE inhibitors and diuretics, with serum creatinine and blood urea nitrogen levels assessed at regular intervals. Electrolyte balance, particularly potassium and sodium levels, requires monitoring during diuretic therapy. Contraindications for specific therapeutic agents include severe bradycardia or atrioventricular conduction disturbances for beta-blockers, hypotension or azotemia for ACE inhibitors, and dehydration or electrolyte imbalances for diuretics. Drug interactions must be considered, particularly the potential for enhanced hypotensive effects when combining ACE inhibitors with diuretics or calcium channel blockers. The therapeutic approach requires individualization based on disease severity, presence of clinical signs, echocardiographic findings, and patient tolerance. Asymptomatic feline patients with mild hypertrophy may require only periodic monitoring, while those with moderate to severe disease benefit from prophylactic therapy to prevent progression to congestive heart failure. Feline patients presenting with acute congestive heart failure require aggressive diuretic therapy, oxygen supplementation, and potential hospitalization for stabilization. Long-term management involves regular reassessment of therapeutic efficacy, dose optimization based on clinical response, and monitoring for disease progression or development of complications. The prognosis varies considerably based on disease severity at diagnosis, with median survival times ranging from months in feline patients with congestive heart failure to years in asymptomatic patients with appropriate management.

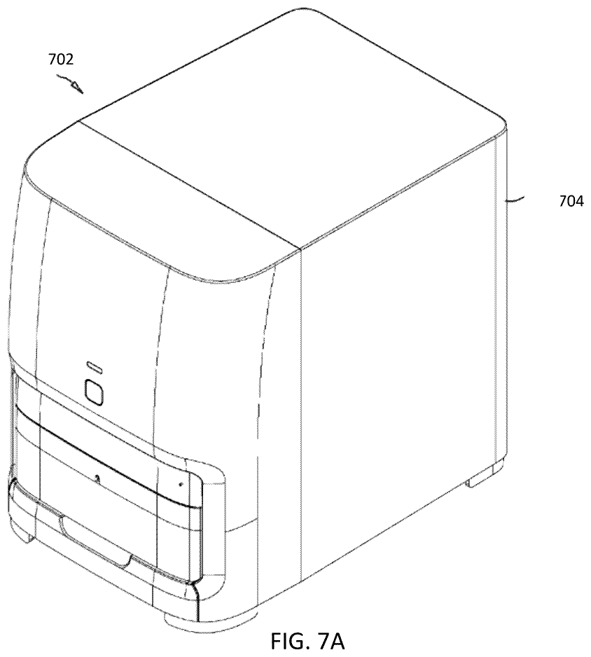

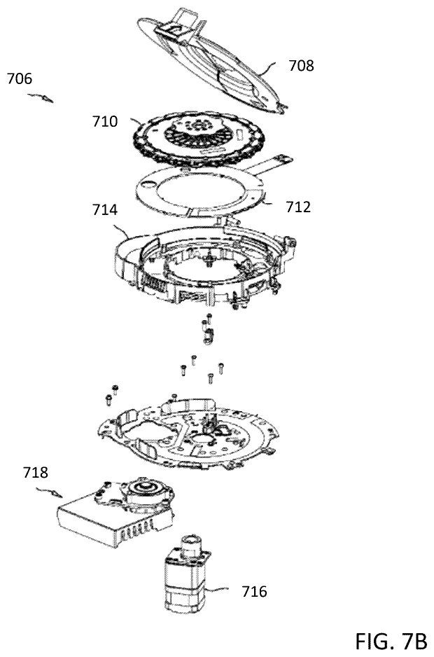

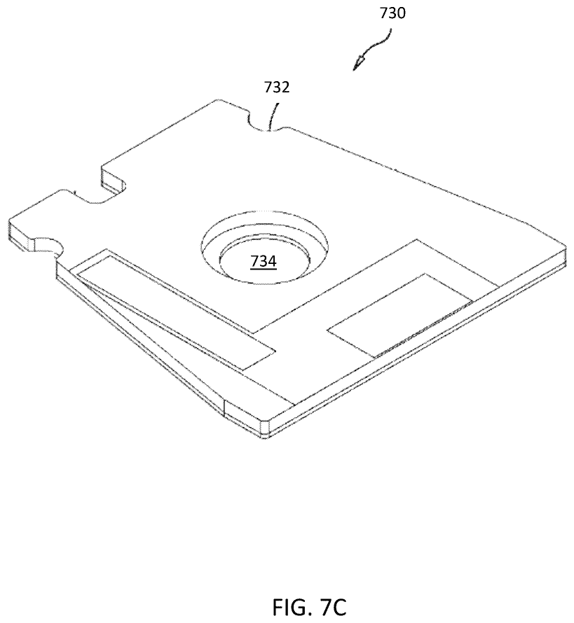

In some embodiments, the computing device systems 100, 200 are communicatively coupled to one or more analyzers. For example and referring to FIGS. 7A and 7B, an example analyzer 702 is schematically depicted. In some embodiments, the analyzer 702 is a chemistry analyzer system including a housing 704. In embodiments, a slide processing assembly 706 is positioned at least partially within the housing 704. The slide processing assembly 706, in embodiments, includes a slide holder 710 and an optics assembly 718. In some embodiments, the slide processing assembly 706 includes a heating element 712, a cover 708, a lower housing 714, and a motor 716. In some embodiments, the slide holder 710 is coupled to the motor 716, such that the motor 716 moves the slide holder 710.

Referring to FIGS. 7B and 7C, in some embodiments, a slide 730 for use in the analyzer 702 (FIG. 7A) is depicted. In some embodiments, the slide 730 includes a slide housing 732 and a film portion 734. In some embodiments, the film portion 734 includes a reagent. Using this slide 730, a predetermined amount of an aqueous sample solution containing a substance to be analyzed (analyte) is placed on a multilayer chemical analysis film of the film portion 734 of the slide 730. The slide 730, in some embodiments, is maintained at a temperature (such as via the heating element 712) for a configurable period of time after which it is irradiated with electromagnetic rays of a wavelength determined in accordance with the analyte and the reagent which is contained in the reagent layer of the multilayer chemical analysis film of the slide 730. Reflection or transmission optical density in a coloration area of the reagent layer is measured (such as via the optics assembly 716) to obtain the concentration of the analyte.

In some embodiments, multiple slides 730 can be positioned in or on the slide holder 710. The slide holder 710, in some embodiments, is movable (such as via the motor 716) such that different slides of the multiple slides 730 can be positioned for interrogation via the optics assembly 716.

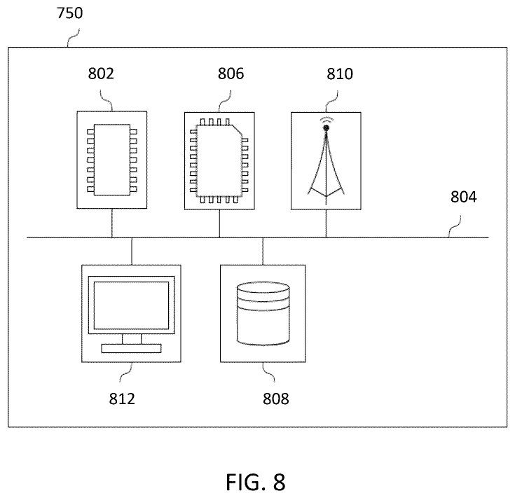

FIG. 8 schematically depicts an example configuration of the control system (ECU) 750 of the analyzer 702 of FIG. 7A. In the illustrated example, the ECU 750 includes one or more processors 802, a communication path 804, one or more memory modules 806, a data storage component 808, network interface hardware 810, and an output device 812.

Each of the one or more processors 802 may be any device capable of executing machine readable and executable instructions. Accordingly, each of the one or more processors 802 may be a controller, an integrated circuit, a microchip, a computer, or any other physical or cloud-based computing device local to or remote from the analyzer 702 (FIG. 7A). The algorithms, including the trained models, signal preprocessing, and noise removal methods discussed below, may be executed by the one or more processors 802. The one or more processors 802 are communicatively coupled to a communication path 804 that provides signal interconnectivity between various modules of the ECU 750. Accordingly, the communication path 804 may communicatively couple any number of processors 802 with one another, and allow the modules coupled to the communication path 804 to operate in a distributed computing environment. Specifically, each of the modules may operate as a node that may send and/or receive data.

The communication path 804 may be formed from any medium that is capable of transmitting a signal such as, for example, conductive wires, conductive traces, optical waveguides, or the like. In some embodiments, the communication path 804 may facilitate the transmission of wireless signals, such as WiFi, Bluetooth®, Near Field Communication (NFC) and the like. Moreover, the communication path 804 may be formed from a combination of mediums capable of transmitting signals. In some embodiments, the communication path 804 comprises a combination of conductive traces, conductive wires, connectors, and buses that cooperate to permit the transmission of electrical data signals to components such as processors, memories, sensors, input devices, output devices, and communication devices. Additionally, it is noted that the term “signal” means a waveform (e.g., electrical, optical, magnetic, mechanical or electromagnetic), such as DC, AC, sinusoidal-wave, triangular-wave, square-wave, vibration, and the like, capable of traveling through a medium.

The ECU 750 includes the one or more memory modules 806 communicatively coupled to the communication path 804. The one or more memory modules 806 may comprise RAM, ROM, flash memories, hard drives, or any device capable of storing machine readable and executable instructions such that the machine readable and executable instructions can be accessed by the one or more processors 802. The machine readable and executable instructions may comprise logic or algorithm(s) written in any programming language of any generation (e.g., 1GL, 2GL, 3GL, 4GL, or 5GL) such as, for example, machine language that may be directly executed by the processor, or assembly language, object-oriented programming (OOP), scripting languages, microcode, etc., that may be compiled or assembled into machine readable and executable instructions and stored on the one or more memory modules 806. Alternatively, the machine readable and executable instructions may be written in a hardware description language (HDL), such as logic implemented via either a field-programmable gate array (FPGA) configuration or an application-specific integrated circuit (ASIC), or their equivalents. Accordingly, the methods described herein may be implemented in any conventional computer programming language, as pre-programmed hardware elements, or as a combination of hardware and software components.

Referring still to FIG. 8, the example ECU 750 includes the data storage component 808. In some embodiments, the data storage component 808 may store data captured by the analyzer 702 (FIG. 7A). The data storage component 808 may also store other data used by the various components of the ECU 750.

In some embodiments, the ECU 750 comprises network interface hardware 410 for communicatively coupling the analyzer 702 (FIG. 3A) to the system 100 (FIG. 1) or the system 200 (FIG. 2). This may allow data to be shared between the devices to improve the data collected by the devices.

In some embodiments, the ECU 750 comprises the output device 812. The output device 812 can include a graphical user interface (GUI), a screen, one or more devices in communication with the one or more processors 802 (such as smartphones, tables, and the like), and/or any other device or interface suitable for displaying data. In some examples, the output device 812, or another device, may be configured to as an input device to receive user input.

While in the example shown in FIGS. 7A-7C, the analyzer 702 is depicted as being a chemistry analyzer, it should be understood that the one or more analyzers may include any suitable analyzer, including and without limitation, a flow cytometer, urine chemistry analyzers, urine sediment analyzers, rapid assay analyzers, and the like. Moreover, while in the embodiment depicted in FIGS. 7A-7C, the analyzer 702 is a slide-based chemistry analyzer, it should be understood that this is merely an example, and the analyzer 702 can be any suitable chemistry analyzer, including and without limitation a rotor-based chemistry analyzer, a rapid assay device, or the like.

Various embodiments of the diagnostic system 300 may be realized by software, or more precisely, an application program running on a microprocessor, or by firmware or hardware implementing the program on the POC terminal and/or the software servers POC. The diagnostic system 300 may include one or more memories which store various data and program modules associated with the diagnostic methods.

It is important to note that the machine learning and diagnostic algorithms described herein do not represent a computer application of the way humans perform diagnoses. Humans interpret new data in the context of everything else they have previously learned. In stark contrast to mental diagnostic processes, artificial intelligence algorithms, and specifically, the machine learning (ML) algorithms described herein, analyze massive data sets to identify patterns and correlations, without understanding any of the data they are processing. This process is fundamentally different from the mental process performed by a veterinarian. Furthermore, the large amounts of data required to the train the machine learning models, and the complexity of the trained models, make it impossible for the algorithms described herein to be performed merely in the human mind.

Accordingly, it should now be understood that concepts of the present disclosure are directed to ensemble learning methods and systems for selecting and training an ensemble of diagnostic models for assessing the risk of feline hypertrophic cardiomyopathy in patients.

Numbered aspects of the present disclosure are provided below:

In a first aspect A1, the present disclosure provides a method, executed by a programmed data processing device system, of receiving first medical training data; training a plurality of first machine learning models on the received first medical training data to generate an ensemble of diagnostic models; receiving second medical training data; training a second machine learning model on the received second medical training data to generate a knowledge based diagnostic model; receiving new patient data; determining whether the ensemble of diagnostic models indicates a risk for feline hypertrophic cardiomyopathy for the new patient data; and, in a case where the ensemble of diagnostic models indicates a risk for feline hypertrophic cardiomyopathy for the new patient data, assessing the risk of feline hypertrophic cardiomyopathy using the knowledge based diagnostic model.

In a second aspect A2, the present disclosure provides the method according to aspect A1, wherein the ensemble of diagnostic models includes one or more of a decision tree model, a random forest model, a neural network, a support vector machine, or a nearest neighbor classifier.

In a third aspect A3, the present disclosure provides the method according to any one of aspects A1-A2, wherein training the plurality of first machine learning models includes selecting at least a first machine learning model and a second machine learning model for the ensemble of diagnostic models, wherein the second machine learning model is different from the first machine learning model; training the first machine learning model using a first subset of the received first medical training data to generate a first model of the ensemble of diagnostic models; and training the second machine learning model using a second subset of the received first medical training data to generate a second model of the ensemble of diagnostic models, wherein the second subset of the received first medical training data is different from the first subset of the received first medical training data, and wherein the risk for feline hypertrophic cardiomyopathy for the new patient data is determined based on outputs of the first machine learning model and the second machine learning model.

In a fourth aspect A4, the present disclosure provides the method according to aspect A3, wherein it is determined that the ensemble of diagnostic models indicates a risk for feline hypertrophic cardiomyopathy for the new patient data in a case where the outputs of either the first machine learning model or the second machine learning indicate the risk for feline hypertrophic cardiomyopathy for the new patient data.

In a fifth aspect A5, the present disclosure provides the method according to any one of aspects A1-A4, wherein training the second machine learning model includes generating an expert model including a plurality of rules based on domain knowledge included in the received second medical training data; refining the plurality of rules in the expert model based on patient data included in the received second medical training data; and generating the knowledge based diagnostic model based on the refined plurality of rules.

In a sixth aspect A6, the present disclosure provides the method according to any one of aspects A1-A5, wherein the knowledge based diagnostic model includes one or more of a rules based model and a data driven machine learning model.

In a seventh aspect A7, the present disclosure provides the method according to any one of aspects A1-A6, wherein assessing the risk of feline hypertrophic cardiomyopathy using the knowledge based diagnostic model further includes displaying, on a user interface, a series of prompts to receive further patient medical data; and assessing the risk of feline hypertrophic cardiomyopathy based the received further patient medical data in response to the series of prompts.

In an eighth aspect A8, the present disclosure provides the method according to aspect A7, wherein each subsequent prompt of the series of prompts is dynamically selected using the knowledge based diagnostic model based on a response to a preceding prompt of the series of prompts.

In a ninth aspect A9, the present disclosure provides the method according to any one of aspects A1-A8, wherein the knowledge based diagnostic model is an interactive model, and wherein assessing the risk of feline hypertrophic cardiomyopathy includes interacting with a user using the knowledge based diagnostic model to provide guidance on diagnosis and treatment of feline hypertrophic cardiomyopathy.

In a tenth aspect A10, the present disclosure provides a diagnostic system for assessing the risk of feline hypertrophic cardiomyopathy, the system comprising a memory configured to store instructions; and a processor communicatively connected to the memory and configured to execute the stored instructions to receive first medical training data; train a plurality of first machine learning models on the received first medical training data to generate an ensemble of diagnostic models; receive second medical training data; train a second machine learning model on the received second medical training data to generate a knowledge based diagnostic model; receive new patient data; determine whether the ensemble of diagnostic models indicates a risk for feline hypertrophic cardiomyopathy for the new patient data; and, in a case where the ensemble of diagnostic models indicates a risk for feline hypertrophic cardiomyopathy for the new patient data, assess the risk of feline hypertrophic cardiomyopathy using the knowledge based diagnostic model.

In an eleventh aspect A11, the present disclosure provides the system according to aspect A10, wherein the ensemble of diagnostic models includes one or more of a decision tree model, a random forest model, a neural network, a support vector machine, or a nearest neighbor classifier.

In a twelfth aspect A12, the present disclosure provides the system according to any one of aspects A10-A11, wherein the plurality of first machine learning models is trained by selecting at least a first machine learning model and a second machine learning model for the ensemble of diagnostic models, wherein the second machine learning model is different from the first machine learning model; training the first machine learning model using a first subset of the received first medical training data to generate a first model of the ensemble of diagnostic models; and training the second machine learning model using a second subset of the received first medical training data to generate a second model of the ensemble of diagnostic models, wherein the second subset of the received first medical training data is different from the first subset of the received first medical training data, and wherein the risk for feline hypertrophic cardiomyopathy for the new patient data is determined based on outputs of the first machine learning model and the second machine learning model.

In a thirteenth aspect A13, the present disclosure provides the system according to aspect A12, wherein it is determined that the ensemble of diagnostic models indicates a risk for feline hypertrophic cardiomyopathy for the new patient data in a case where the outputs of either the first machine learning model or the second machine learning indicate the risk for feline hypertrophic cardiomyopathy for the new patient data.

In a fourteenth aspect A14, the present disclosure provides the system according to any one of aspects A10-A13, wherein the second machine learning model is trained by generating an expert model including a plurality of rules based on domain knowledge included in the received second medical training data; refining the plurality of rules in the expert model based on patient data included in the received second medical training data; and generating the knowledge based diagnostic model based on the refined plurality of rules.

In a fifteenth aspect A15, the present disclosure provides the system according to any one of aspects A10-A14, wherein the knowledge based diagnostic model includes one or more of a rules based model and a data driven machine learning model.

In a sixteenth aspect A16, the present disclosure provides the system according to any one of aspects A10-A15, wherein the risk of feline hypertrophic cardiomyopathy is assessed using the knowledge based diagnostic model by displaying, on a user interface, a series of prompts to receive further patient medical data; and assessing the risk of feline hypertrophic cardiomyopathy based the received further patient medical data in response to the series of prompts.

In a seventeenth aspect A17, the present disclosure provides the system according to aspect A16, wherein each subsequent prompt of the series of prompts is dynamically selected using the knowledge based diagnostic model based on a response to a preceding prompt of the series of prompts.

In an eighteenth aspect A18, the present disclosure provides the system according to any one of aspects A10-A17, wherein the knowledge based diagnostic model is an interactive model, and wherein the processor is configured to further execute the stored instructions to interact with a user using the knowledge based diagnostic model to provide guidance on diagnosis and treatment of feline hypertrophic cardiomyopathy.

In a nineteenth aspect A19, the present disclosure provides non-transitory computer readable storage medium configured to store a program that executes a diagnostic method, the method comprising receiving first medical training data; training a plurality of first machine learning models on the received first medical training data to generate an ensemble of diagnostic models; receiving second medical training data; training a second machine learning model on the received second medical training data to generate a knowledge based diagnostic model; receiving new patient data; determining whether the ensemble of diagnostic models indicates a risk for feline hypertrophic cardiomyopathy for the new patient data; and, in a case where the ensemble of diagnostic models indicates a risk for feline hypertrophic cardiomyopathy for the new patient data, assessing the risk of feline hypertrophic cardiomyopathy using the knowledge based diagnostic model.

In a twentieth aspect A20, the present disclosure provides a method, executed by a programmed data processing device system, of assessing the risk of feline hypertrophic cardiomyopathy, the method comprising receiving new patient data; receiving a machine-learning based diagnostic model and a knowledge based diagnostic model; determining whether the machine-learning based diagnostic model indicates a risk for feline hypertrophic cardiomyopathy for the new patient data; and, in a case where the machine-learning based diagnostic model indicates a risk for feline hypertrophic cardiomyopathy for the new patient data, assessing the risk of feline hypertrophic cardiomyopathy using the knowledge based diagnostic model.

In a twenty-first aspect A21, the present disclosure provides the method according to aspect A20, wherein the machine-learning based diagnostic model includes one or more of a decision tree model, a random forest model, a neural network, a support vector machine, or a nearest neighbor classifier.

In a twenty-second aspect A22, the present disclosure provides the method according to any one of aspects A20-A21, wherein the method further comprises receiving first training data; and training the machine-learning based diagnostic model by selecting at least a first machine learning model and a second machine learning model, wherein the second machine learning model is different from the first machine learning model; training the first machine learning model using a first subset of the received first training data to generate a first diagnostic model; training the second machine learning model using a second subset of the received first training data to generate a second diagnostic model; and combining the first diagnostic model and the second diagnostic model into an ensemble diagnostic model to be used as the machine-learning based diagnostic model, wherein the risk for feline hypertrophic cardiomyopathy for the new patient data is determined based on an output of the ensemble diagnostic model.

In a twenty-third aspect A23, the present disclosure provides the method according to aspect A22, wherein it is determined that the ensemble diagnostic model indicates a risk for feline hypertrophic cardiomyopathy for the new patient data in a case where the outputs of either the first diagnostic model or the second diagnostic model indicate the risk for feline hypertrophic cardiomyopathy for the new patient data.

In a twenty-fourth aspect A24, the present disclosure provides the method according to any one of aspects A20-A23, wherein the method further comprises receiving second training data; and training the knowledge based diagnostic model by generating an expert model including a plurality of rules based on domain knowledge included in the received second medical training data; and refining the plurality of rules in the expert model based on patient data included in the received second medical training data.

In a twenty-fifth aspect A25, the present disclosure provides the method according to any one of aspects A20-A24, wherein the knowledge based diagnostic model includes one or more of a rule based model or a data driven machine learning model.

In a twenty-sixth aspect A26, the present disclosure provides the method according to any one of aspects A20-A25, wherein assessing the risk of feline hypertrophic cardiomyopathy using the knowledge based diagnostic model further includes displaying, on a user interface, a series of prompts to receive further patient medical data; and assessing the risk of feline hypertrophic cardiomyopathy based the received further patient medical data in response to the series of prompts.

In a twenty-seventh aspect A27, the present disclosure provides the method according to aspect A26, wherein each subsequent prompt of the series of prompts is dynamically selected using the knowledge based diagnostic model based on a response to a preceding prompt of the series of prompts.

In a twenty-eighth aspect A28, the present disclosure provides the method according to any one of aspects A20-A27,wherein the knowledge based diagnostic model is an interactive model, and wherein assessing the risk of feline hypertrophic cardiomyopathy includes interacting with a user using the knowledge based diagnostic model to provide guidance on diagnosis and treatment of feline hypertrophic cardiomyopathy.

In a twenty-ninth aspect A29, the present disclosure provides a diagnostic system for assessing the risk of feline hypertrophic cardiomyopathy, the system comprising a memory configured to store instructions; and a processor communicatively connected to the memory and configured to execute the stored instructions to receive new patient data; receive a machine-learning based diagnostic model and a knowledge based diagnostic model; determine whether the machine-learning based diagnostic model indicates a risk for feline hypertrophic cardiomyopathy for the new patient data; and, in a case where the machine-learning based diagnostic model indicates a risk for feline hypertrophic cardiomyopathy for the new patient data, assess the risk of feline hypertrophic cardiomyopathy using the knowledge based diagnostic model.

In a thirtieth aspect A30, the present disclosure provides the system according to aspect A29, wherein the machine-learning based diagnostic model includes one or more of a decision tree model, a random forest model, a neural network, a support vector machine, or a nearest neighbor classifier.

In a thirty-first aspect A31, the present disclosure provides the system according to any one of aspects A29-30, wherein the processor is further configured to execute the stored instructions to receive first training data; and train the machine-learning based diagnostic model by selecting at least a first machine learning model and a second machine learning model, wherein the second machine learning model is different from the first machine learning model; training the first machine learning model using a first subset of the received first training data to generate a first diagnostic model; training the second machine learning model using a second subset of the received first training data to generate a second diagnostic model; and combining the first diagnostic model and the second diagnostic model into an ensemble diagnostic model to be used as the machine-learning based diagnostic model, and wherein the risk for feline hypertrophic cardiomyopathy for the new patient data is determined based on an output of the ensemble diagnostic model.

In a thirty-second aspect A32, the present disclosure provides the system according to aspect A31, wherein it is determined that the ensemble diagnostic model indicates a risk for feline hypertrophic cardiomyopathy for the new patient data in a case where the outputs of either the first diagnostic model or the second diagnostic model indicate the risk for feline hypertrophic cardiomyopathy for the new patient data.

In a thirty-third aspect A33, the present disclosure provides the system according to any one of aspects A29-32, wherein the processor is further configured to execute the stored instructions to receive second training data; and train the knowledge based diagnostic model by generating an expert model including a plurality of rules based on domain knowledge included in the received second medical training data; and refining the plurality of rules in the expert model based on patient data included in the received second medical training data.

In a thirty-fourth aspect A34, the present disclosure provides the system according to any one of aspects A29-33, wherein the knowledge based diagnostic model includes one or more of a rule based model or a data driven machine learning model.

In a thirty-fifth aspect A35, the present disclosure provides the system according to any one of aspects A29-34, wherein the risk of feline hypertrophic cardiomyopathy is assessed using the knowledge based diagnostic model by displaying, on a user interface, a series of prompts to receive further patient medical data; and assessing the risk of feline hypertrophic cardiomyopathy based the received further patient medical data in response to the series of prompts.

In a thirty-sixth aspect A36, the present disclosure provides the system according to aspect A35, wherein each subsequent prompt of the series of prompts is dynamically selected using the knowledge based diagnostic model based on a response to a preceding prompt of the series of prompts.

In a thirty-seventh aspect A37, the present disclosure provides the system according to any one of aspects A29-36, wherein the knowledge based diagnostic model is an interactive model, and wherein the processor is configured to further execute the stored instructions to interact with a user using the knowledge based diagnostic model to provide guidance on diagnosis and treatment of feline hypertrophic cardiomyopathy.

In a thirty-eighth aspect A38, the present disclosure provides a non-transitory computer readable storage medium configured to store a program that executes a diagnostic method, the method comprising receiving new patient data; receiving a machine-learning based diagnostic model and a knowledge based diagnostic model; determining whether the machine-learning based diagnostic model indicates a risk for feline hypertrophic cardiomyopathy for the new patient data; and, in a case where the machine-learning based diagnostic model indicates a risk for feline hypertrophic cardiomyopathy for the new patient data, assessing the risk of feline hypertrophic cardiomyopathy using the knowledge based diagnostic model.

Subsets or combinations of various embodiments described above provide further embodiments.

These and other changes can be made to the invention in light of the above-detailed description and still fall within the scope of the present invention. In general, in the following claims, the terms used should not be construed to limit the invention to the specific embodiments disclosed in the specification. Accordingly, the invention is not limited by the disclosure, but instead its scope is to be determined entirely by the following claims.

What is claimed is:

1. A processor executed method for assessing the risk of feline hypertrophic cardiomyopathy, comprising:
receiving first medical training data;
training a plurality of first machine learning models on the received first medical training data to generate an ensemble of diagnostic models;
receiving second medical training data;
training a second machine learning model on the received second medical training data to generate a knowledge based diagnostic model;
receiving new patient data;
determining whether the ensemble of diagnostic models indicates a risk for feline hypertrophic cardiomyopathy for the new patient data; and
in a case where the ensemble of diagnostic models indicates a risk for feline hypertrophic cardiomyopathy for the new patient data, assessing the risk of feline hypertrophic cardiomyopathy using the knowledge based diagnostic model.

2. The method according to claim 1, wherein the ensemble of diagnostic models includes one or more of a decision tree model, a random forest model, a neural network, a support vector machine, or a nearest neighbor classifier.

3. The method according to claim 1,
wherein training the plurality of first machine learning models includes:
selecting at least a first machine learning model and a second machine learning model for the ensemble of diagnostic models, wherein the second machine learning model is different from the first machine learning model;
training the first machine learning model using a first subset of the received first medical training data to generate a first model of the ensemble of diagnostic models; and
training the second machine learning model using a second subset of the received first medical training data to generate a second model of the ensemble of diagnostic models, wherein the second subset of the received first medical training data is different from the first subset of the received first medical training data, and
wherein the risk for feline hypertrophic cardiomyopathy for the new patient data is determined based on outputs of the first machine learning model and the second machine learning model.

4. The method according to claim 3, wherein, it is determined that the ensemble of diagnostic models indicates a risk for feline hypertrophic cardiomyopathy for the new patient data in a case where the outputs of either the first machine learning model or the second machine learning indicate the risk for feline hypertrophic cardiomyopathy for the new patient data.

5. The method according to claim 1,
wherein training the second machine learning model includes:
generating an expert model including a plurality of rules based on domain knowledge included in the received second medical training data;
refining the plurality of rules in the expert model based on patient data included in the received second medical training data; and
generating the knowledge based diagnostic model based on the refined plurality of rules.

6. The method according to claim 1, wherein the knowledge based diagnostic model includes one or more of a rules based model and a data driven machine learning model.

7. The method according to claim 1,
wherein assessing the risk of feline hypertrophic cardiomyopathy using the knowledge based diagnostic model further includes:
displaying, on a user interface, a series of prompts to receive further patient medical data; and
assessing the risk of feline hypertrophic cardiomyopathy based the received further patient medical data in response to the series of prompts.

8. The method according to claim 7, wherein each subsequent prompt of the series of prompts is dynamically selected using the knowledge based diagnostic model based on a response to a preceding prompt of the series of prompts.

9. The method according to claim 1,
wherein the knowledge based diagnostic model is an interactive model, and
wherein assessing the risk of feline hypertrophic cardiomyopathy includes interacting with a user using the knowledge based diagnostic model to provide guidance on diagnosis and treatment of feline hypertrophic cardiomyopathy.

10. A diagnostic system for assessing the risk of feline hypertrophic cardiomyopathy, the system comprising:
a memory configured to store instructions; and
a processor communicatively connected to the memory and configured to execute the stored instructions to:
receive first medical training data;
train a plurality of first machine learning models on the received first medical training data to generate an ensemble of diagnostic models;
receive second medical training data;
train a second machine learning model on the received second medical training data to generate a knowledge based diagnostic model;
receive new patient data;
determine whether the ensemble of diagnostic models indicates a risk for feline hypertrophic cardiomyopathy for the new patient data; and
in a case where the ensemble of diagnostic models indicates a risk for feline hypertrophic cardiomyopathy for the new patient data, assess the risk of feline hypertrophic cardiomyopathy using the knowledge based diagnostic model.

11. The system according to claim 10, wherein the ensemble of diagnostic models includes one or more of a decision tree model, a random forest model, a neural network, a support vector machine, or a nearest neighbor classifier.

12. The system according to claim 10,
the plurality of first machine learning models is trained by:
selecting at least a first machine learning model and a second machine learning model for the ensemble of diagnostic models, wherein the second machine learning model is different from the first machine learning model;
training the first machine learning model using a first subset of the received first medical training data to generate a first model of the ensemble of diagnostic models; and
training the second machine learning model using a second subset of the received first medical training data to generate a second model of the ensemble of diagnostic models, wherein the second subset of the received first medical training data is different from the first subset of the received first medical training data, and
wherein the risk for feline hypertrophic cardiomyopathy for the new patient data is determined based on outputs of the first machine learning model and the second machine learning model.

13. The system according to claim 12, wherein, it is determined that the ensemble of diagnostic models indicates a risk for feline hypertrophic cardiomyopathy for the new patient data in a case where the outputs of either the first machine learning model or the second machine learning indicate the risk for feline hypertrophic cardiomyopathy for the new patient data.

14. The system according to claim 10,
wherein the second machine learning model is trained by:
generating an expert model including a plurality of rules based on domain knowledge included in the received second medical training data;
refining the plurality of rules in the expert model based on patient data included in the received second medical training data; and
generating the knowledge based diagnostic model based on the refined plurality of rules.

15. The system according to claim 10, wherein the knowledge based diagnostic model includes one or more of a rules based model and a data driven machine learning model.

16. The system according to claim 10,
wherein the risk of feline hypertrophic cardiomyopathy is assessed using the knowledge based diagnostic model by:
displaying, on a user interface, a series of prompts to receive further patient medical data; and
assessing the risk of feline hypertrophic cardiomyopathy based the received further patient medical data in response to the series of prompts.

17. The system according to claim 16, wherein each subsequent prompt of the series of prompts is dynamically selected using the knowledge based diagnostic model based on a response to a preceding prompt of the series of prompts.

18. The system according to claim 10,
wherein the knowledge based diagnostic model is an interactive model, and
wherein the processor is configured to further execute the stored instructions to interact with a user using the knowledge based diagnostic model to provide guidance on diagnosis and treatment of feline hypertrophic cardiomyopathy.

19. A non-transitory computer readable storage medium configured to store a program that executes a diagnostic method, the method comprising:
receiving first medical training data;
training a plurality of first machine learning models on the received first medical training data to generate an ensemble of diagnostic models;
receiving second medical training data;
training a second machine learning model on the received second medical training data to generate a knowledge based diagnostic model;
receiving new patient data;
determining whether the ensemble of diagnostic models indicates a risk for feline hypertrophic cardiomyopathy for the new patient data; and
in a case where the ensemble of diagnostic models indicates a risk for feline hypertrophic cardiomyopathy for the new patient data, assessing the risk of feline hypertrophic cardiomyopathy using the knowledge based diagnostic model.
